(19) United States
(12) Patent Application Publication
(10) Pub. No.: US 2024/0345074 A1
(43) Pub. Date: Oct. 17, 2024

(54) BIOSENSORS FOR THE DETECTION OF ANTIGENS USING ANTIBODY FRAGMENTS

(71) Applicant: University of Pittsburgh - Of the Commonwealth System of Higher Education, Pittsburgh, PA (US)

(72) Inventors: Alexander Star, Pittsburgh, PA (US); Wenting Shao, Pittsburgh, PA (US)

(21) Appl. No.: 18/631,488

(22) Filed: Apr. 10, 2024

Related U.S. Application Data
(60) Provisional application No. 63/459,353, filed on Apr. 14, 2023.

(51) Int. Cl. G01N 33/543; G01N 27/327; G01N 27/414; G01N 33/487

(52) U.S. Cl. CPC G01N 33/54346; G01N 27/3278; G01N 27/4145; G01N 27/4146; G01N 33/48714; G01N 2800/307

(57) ABSTRACT

A sensor device for detecting an antigen in a fluid sample includes a sensor including a substrate and a sensor medium on the substrate. The sensor medium includes a plurality of single-walled carbon nanotubes having an enriched semiconducting content and one or more antibody fragments immobilized on the plurality of single-walled carbon nanotubes. Each of the one or more antibody fragments includes an active binding site for the antigen. The sensor device further includes electronic circuitry including at least one measurement system in operative connection with the sensor to measure a variable providing a measure of change in at least one property of the sensor medium which is dependent upon the presence of the antigen.

### CROSS-REFERENCE TO RELATED APPLICATIONS

This application claims benefit of U.S. Provisional Patent Application Ser. No. 63/459,353, filed Apr. 14, 2023, the disclosure of which is incorporated herein by reference.

### GOVERNMENTAL INTEREST

This invention was made with government support under grant number HDTRA1-21-1-0009 awarded by the Defense Threat Reduction Agency of the Department of Defense. The government has certain rights in the invention.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited herein are incorporated by reference.

The opioid crisis is a worldwide public health crisis that has affected millions of people. In recent years, synthetic opioids, primarily illicit fentanyl, have become the primary driver of overdose deaths. There is a great need for a highly sensitive, portable and inexpensive analytical tool that can quickly indicate the presence and relative threat of fentanyl.

Fentanyl (N-phenyl-N-[1-(2-phenylethyl)piperidinyl]-propanamide), which is approximately 50-100 times more potent than morphine, is a potent synthetic opioid that is used as a pain reliever and as an anesthetic. However, due to its pharmacological effects, an overdose of fentanyl can cause difficulties in breathing, and can lead to death. According to the US Centers for Disease Control and Prevention (CDC), synthetic opioids are the primary driver of overdose deaths in the United States, making opioid overdose deaths a major public health crisis. Moreover, fentanyl can also be found in combination with other drugs, such as heroin or cocaine.

In the human body, fentanyl is rapidly metabolized in the liver to norfentanyl via oxidative N-dealkylation and to 4-anilino-N-phenethylpiperidine (4-ANPP) via hydrolysis. Norfentanyl, as the primary inactive metabolite of fentanyl, can also be detected in body fluids, such as urine and blood, and the level of norfentanyl is sometimes tested in people who have been prescribed fentanyl or other opioids, or in individuals who may have used or been exposed to these drugs. Testing for norfentanyl can be more reliable and accurate, particularly in cases where the sample may be degraded, or the pH of the urine is not optimal. In addition, norfentanyl has a longer detection window, thus testing for norfentanyl can provide more information about an individual's past exposure to fentanyl.

Instrumental analytical methods, such as gas chromatography-mass spectrometry (GC-MS) and liquid chromatography-tandem mass spectrometry (LC-MS/MS) are the most commonly used techniques for the detection of norfentanyl. Laboratories often combine chromatographic methods with immunoassays to ensure both high sensitivity and specificity. However, these techniques require specialized equipment and training, making them relatively expensive and time-consuming. Meanwhile, as a result of the lack of redox activity of norfentanyl, certain electrochemical methods, although are generally considered to be rapid, simple, and low-cost, are not suitable for norfentanyl detection.

Carbon nanomaterial-based field-effect transistor (FET) biosensors have shown remarkable sensitivity and low detection limits for a variety of biological analytes. Previously, an aptamer-based graphene FET (AptG-FET) platform was reported for the simultaneous detection of three different opioid metabolites in wastewater. The AptG-FET platform with a coplanar Pt gate enabled multianalyte detection on a single chip and achieved pg/mL level limit of detection for noroxycodone (NX), 2-ethylidene-1,5-dimethyl-3,3-diphenylpyrrolidine (EDDP), and norfentanyl. Semiconductor-enriched (sc-) single-walled carbon nanotubes (SWCNTs), on the other hand, are particularly promising candidates for FET biosensors because the high-purity semiconducting content offers high on/off ratio for FETs, facilitating ultrasensitivity of sc-SWCNT-based FET biosensors. Additionally, because of the presence of functional groups on the sidewalls, SWCNTs provide more avenues for functionalization with custom-designed chemistry to preferentially interact with target biomolecules, which leads to excellent sensitivity in complex media, such as saliva, sweat, and serum.

US Patent Application Publication No. US2022/0365078, the disclosure of which is incorporated herein by reference, discloses methods of detecting an antigen analyte in which antibodies are immobilized on a sensor medium including nanostructures such as semiconductor-enriched SWCNTs to sense antigens including opioids. Norfentanyl was sensed using fentanyl antibody-functionalized semiconductor-enriched SWCNT FET sensors.

There is a great need for improved highly sensitive, portable and inexpensive analytical devices and methodologies that can quickly indicate the presence and relative threat of fentanyl.

### SUMMARY

In one aspect, a sensor device for detecting an antigen in a fluid sample includes a sensor including a substrate and a sensor medium on the substrate. The sensor medium includes a plurality of single-walled carbon nanotubes having an enriched semiconducting content and one or more antibody fragments immobilized on the plurality of single-walled carbon nanotubes. In a number of embodiments, the semiconducting content is at least 90%, at least 95%, at least 99%, or at least 99.9%. Each of the one or more antibody fragments includes an active binding site for the antigen. The one or more antibody fragments may, for example, include reduced antibody fragments. The sensor device further includes electronic circuitry including at least one measurement system in operative connection with the sensor to measure a variable providing a measure of change in at least one property of the sensor medium which is dependent upon the presence of the antigen. The variable measured may be an electrical property change. The at least one measurement system may, for example, include a calibrated algorithm to determine a concentration of the antigen based on a value of the variable.

The one or more antibody fragments may, for example, be covalently attached to at least a portion of the plurality of single-walled carbon nanotubes or be attached to at least a portion of a plurality of noble metal (for example, gold) nanoparticles immobilized upon the plurality of single-walled carbon nanotubes. In a number of embodiments, the one or more antibody fragments are attached to at least a portion of the plurality of gold nanoparticles immobilized upon the plurality of single-walled carbon nanotubes.

In a number of embodiments, the antigen is a drug or a metabolite of a drug. In a number of embodiments, the drug is an opioid. Examples of antigen drugs include fentanyl, cocaine, morphine, hydrocodone, codeine, and tetrahydrocannabinol (THC). Examples of metabolites of drugs include norfentanyl and 6-Acetylmorphine.

In a number of embodiments, the sensor is incorporated within a field effect transistor circuit of the electronic circuitry. In a number of embodiments, a liquid of known and low ionic strength may be used as a liquid gate. Ionic strength can also be expressed as conductivity or resistivity. The liquid can, for example, be a purified water such as nanopure water or a diluted phosphate buffered saline (PBS). In embodiments in which the sensor is incorporated within a field effect transistor circuit of the electronic circuitry, the field effect transistor circuitry may include a gold gate, and the antibody fragments are also immobilized on the gold gate.

A plurality of the sensors may be included in the sensor devices hereof. In a number of embodiments, the sensor medium is maintained in (or in fluid connection with) a liquid phase.

In another aspect, a sensor includes a substrate and a sensor medium on the substrate. The sensor medium includes a plurality of single-walled carbon nanotubes having an enriched semiconducting content and one or more antibody fragments immobilized on the plurality of single-walled carbon nanotubes. In a number of embodiments, the semiconducting content is at least 90%, at least 95%, at least 99%, or at least 99.9%. Each of the one or more antibody fragments includes an active binding site for an antigen to be detected. The one or more antibody fragments may, for example, include reduced antibody fragments. The sensor device further includes electronic circuitry including at least one measurement system in operative connection with the sensor to measure a variable providing a measure of change in at least one property of the sensor medium which is dependent upon the presence of the antigen. The variable measured may be an electrical property change.

As described above, the one or more antibody fragments may, for example, be covalently attached to at least a portion of the plurality of single-walled carbon nanotubes or be attached to at least a portion of a plurality of gold nanoparticles immobilized upon the plurality of single-walled carbon nanotubes. In a number of embodiments, the one or more antibody fragments are attached to at least a portion of the plurality of gold nanoparticles immobilized upon the plurality of single-walled carbon nanotubes.

The antigen may be a drug or a metabolite of a drug. In a number of embodiments, the drug is an opioid. Examples of antigen drugs include fentanyl, cocaine, morphine, hydrocodone, codeine, and tetrahydrocannabinol (THC). Examples of metabolites of drugs include norfentanyl and 6-Acetylmorphine.

In another aspect, a method of detecting an antigen in a fluid sample includes providing a sensor device hereof, exposing the sensor of the sensor device to the fluid sample for a period of time; and measuring an output of the sensor. The output of the sensor may be measured with a liquid of known ionic strength over the sensor medium. In a number of embodiments, subsequent to exposing the sensor to the fluid sample, the sensor is washed one or more times with a liquid of known ionic strength, and measuring the output of the sensor is performed after washing the sensor with the liquid of known ionic strength over the sensor medium. The ionic strength of the liquid may be chosen to be less than that of the fluid sample to increase sensitivity compared to an output measured in the presence of the fluid sample. In a number of embodiments, the liquid is a purified water. The purified water has a resistivity greater than or equal to 18.2 MO-cm in a number of embodiments.

As described above, the one or more antibody fragments may, for example, be covalently attached to at least a portion of the plurality of single-walled carbon nanotubes or be attached to at least a portion of a plurality of gold nanoparticles immobilized upon the plurality of single-walled carbon nanotubes. In a number of embodiments, the one or more antibody fragments are attached to at least a portion of the plurality of gold nanoparticles immobilized upon the plurality of single-walled carbon nanotubes.

As also described above, the antigen may be a drug or a metabolite of a drug. In a number of embodiments, the drug is an opioid. Examples of antigen drugs include fentanyl, cocaine, morphine, hydrocodone, codeine, and tetrahydrocannabinol (THC). Examples of metabolites of drugs include norfentanyl and 6-Acetylmorphine.

In another aspect, a composition or composite composition includes a plurality of single-walled carbon nanotubes having an enriched semiconducting content and one or more antibody fragments immobilized on the plurality of single-walled carbon nanotubes. In a number of embodiments, the semiconducting content is at least 90%, at least 95%, at least 99%, or at least 99.9%. Each of the one or more antibody fragments includes an active binding site for an antigen. The one or more antibody fragments may, for example, include reduced antibody fragments.

The one or more antibody fragments may, for example, be covalently attached to at least a portion of the plurality of single-walled carbon nanotubes or be attached to at least a portion of a plurality of gold nanoparticles immobilized upon the plurality of single-walled carbon nanotubes. In a number of embodiments, the one or more antibody fragments are attached to at least a portion of the plurality of gold nanoparticles immobilized upon the plurality of single-walled carbon nanotubes.

In a further aspect, a method of forming a sensor includes immobilizing one or more antibody fragments immobilized on a plurality of single-walled carbon nanotubes of a composition includes the plurality of single-walled carbon nanotubes to form a sensor medium. The single-walled carbon nanotubes having an enriched semiconducting content. In a number of embodiments, the semiconducting content is at least 90%, at least 95%, at least 99%, or at least 99.9%. Each of the one or more antibody fragments includes an active binding site for an antigen to be detected by the sensor. The one or more antibody fragments may, for example, include reduced antibody fragments.

In a further aspect, a method of detecting an antigen which is a drug or a drug metabolite in a fluid sample includes providing a sensor device including a sensor including a substrate and a sensor medium on the substrate. The sensor medium includes a plurality of nanostructures having an enriched semiconducting content and one or more antibody fragments. Each of the one or more antibody fragments includes an active binding site for the antigen. In a number of embodiments, the semiconducting content is at least 90%, at least 95%, at least 99%, or at least 99.9%. Each of the one or more antibody fragments includes an active binding site. The one or more antibody fragments may, for example, include reduced antibody fragments. The sensor device further include electronic circuitry including at least one measurement system in operative connection with the sensor to measure a variable providing a measure of change in at least one property of the sensor medium which is dependent upon the presence of the antigen, exposing the sensor to the fluid sample for a period of time; and measuring an output of the sensor.

The one or more antibody fragments may, for example, be covalently attached to at least a portion of the plurality of single-walled carbon nanotubes or be attached to at least a portion of a plurality of gold nanoparticles immobilized upon the plurality of single-walled carbon nanotubes. In a number of embodiments, the one or more antibody fragments are attached to at least a portion of the plurality of gold nanoparticles immobilized upon the plurality of single-walled carbon nanotubes.

In a further aspect, a sensor device for detecting an antigen which is an opioid or a metabolite of an opioid in a fluid sample includes a sensor including a substrate and a sensor medium on the substrate. The sensor medium includes a plurality of nanostructures and one or more antibody fragments immobilized on the plurality of nanostructures. The one or more antibody fragments may, for example, include reduced antibody fragments. The sensor device further includes electronic circuitry including at least one measurement system in operative connection with the sensor to measure a variable providing a measure of change in at least one property of the sensor medium which is dependent upon the presence of the antigen. The opioid may, for example, be fentanyl. The metabolite of an opioid may, for example, be norfentanyl.

In still a further aspect, a sensor includes a substrate and a sensor medium on the substrate. The sensor medium includes a plurality of nanostructures and one or more antibody fragments immobilized on the plurality of single-walled carbon nanotubes. The antibody fragments are fragments of antibodies for an antigen which is an opioid or a metabolite of an opioid. Each of the one or more antibody fragments includes an active binding site for the antigen. The one or more antibody fragments may, for example, include reduced antibody fragments. Electronic circuitry including at least one measurement system may be in operative connection with the sensor to measure a variable providing a measure of change in at least one property of the sensor medium which is dependent upon the presence of the antigen. The variable measured may be an electrical property change. The opioid may, for example, be fentanyl. The metabolite of an opioid may, for example, be norfentanyl.

In another aspect, a method of detecting an antigen which is an opioid or a metabolite of an opioid in a fluid sample includes providing a sensor device hereof, exposing the sensor of the sensor device to the fluid sample for a period of time; and measuring an output of the sensor. As described above, the sensor may include a substrate and a sensor medium on the substrate. The sensor medium includes a plurality of nanostructures and one or more antibody fragments immobilized on the plurality of single-walled carbon nanotubes. The antibody fragments are fragments of antibodies for the antigen. Each of the one or more antibody fragments includes an active binding site for the antigen. The one or more antibody fragments may, for example, include reduced antibody fragments. The sensor device further includes electronic circuitry including at least one measurement system in operative connection with the sensor to measure a variable providing a measure of change in at least one property of the sensor medium which is dependent upon the presence of the antigen. The variable measured may be an electrical property change. The opioid may, for example, be fentanyl. The metabolite of an opioid may, for example, be norfentanyl.

The present devices, systems, methods, and compositions along with the attributes and attendant advantages thereof, will best be appreciated and understood in view of the following detailed description taken in conjunction with the accompanying drawings.

### BRIEF DESCRIPTION OF THE DRAWINGS

FIG. 1A illustrates a norfentanyl-antibody-functionalized SWCNT-based FET biosensor wherein the upper portion of the figure provides an optical image of the sensing chip with 8 devices, and a bottom portion of the figure illustrates schematically an embodiment of a sensing chip which was wire-bonded in a package for measurements.

FIG. 1B illustrates schematically an embodiment of a norfentanyl-antibody-functionalized SWCNT-based FET biosensor wherein the antibodies are attached via direct coupling approach.

FIG. 1C illustrates FET transfer characteristics of each functionalization step using the direct coupling approach.

FIG. 1D illustrates schematically and embodiment of a norfentanyl-antibody-functionalized SWCNT-based FET biosensor wherein the antibodies are attached via a gold nanoparticle (AuNP) coupling approach.

FIG. 1E illustrates FET transfer characteristics of each functionalization step using AuNP approach.

FIG. 1F illustrates a schematic representation of embodiment of an FET sensor device hereof.

FIG. 1G illustrates a schematic representation of an embodiment of a chemiresistor sensor device hereof.

FIG. 2A illustrates a side-by-side comparison of binding of a whole IgG antibody and a reduced IgG antibody fragment (sometimes referred to herein as a “reduced antibody”) on AuNPs deposited upon sc-SWCNTs.

FIG. 2B illustrates norfentanyl sensing in PBS and synthetic urine using reduced norfentanyl ab-Au-sc-SWCNT devices.

FIG. 3A illustrates a full IgG antibody.

FIG. 3B illustrates the F(ab′)2 (˜110 kDa) antibody fragment which contains two antigen-binding sites joined at the hinge through disulfides.

FIG. 3C illustrates the Fab (˜50 kDa) antibody fragment, which is a monovalent fragment including a light chain and a portion of the heavy chain.

FIG. 3D illustrates the Fab′ (˜55 kDa) antibody fragment which included the variable domains of the heavy and light chains of the antibody, as well as a portion of the hinge region (and it may also contain small portion of Fc).

FIG. 3E illustrates the Fv (˜25 k Da) antibody fragment, which is the smallest fragment produced from IgG that contains a complete antigen-binding site.

FIG. 3F illustrates the reduced IgG (“r-IgG”) antibody fragment, which is produced by using a mild reducing reagent that can selectively cleave the disulfide bridges in the hinge region of an IgG antibody.

FIG. 4A illustrates the D and G peak regions of Raman spectra of the SWCNTs before and after the immobilization of norfentanyl antibody, wherein the Raman spectra were recorded using a 638 nm excitation laser, and all spectra were normalized to the G peak at 1587 cm⁻¹.

FIG. 4B illustrates the Raman spectra were recorded using a 785 nm excitation laser. All spectra were normalized to the Si peak at 507 cm⁻¹ (not shown).

FIG. 4C illustrates Raman spectra of the FET device during each functionalization step using the AuNP approach, wherein the Raman spectra were recorded using a 638 nm excitation laser, and all spectra were normalized to the Si peak at 507 cm⁻¹ (denoted by the asterisk).

FIG. 5A illustrates norfentanyl sensing performance in PBS using ab-sc-SWCNT devices (direct coupling approach), wherein the inset shows FET transfer characteristics of norfentanyl ab-sc-SWCNT devices upon adding increasing concentrations of norfentanyl, and the direction of the arrow in the inset represents increasing concentration across the data lines from 1 fg/mL to 1 μg/mL.

FIG. 5B illustrates norfentanyl sensing performance in PBS using norfentanyl ab-Au-sc-SWCNT devices (AuNP approach), wherein the inset shows FET transfer characteristics of norfentanyl ab-Au-sc-SWCNT devices upon adding decreasing concentrations of norfentanyl, and the direction of the arrow in the inset represents decreasing concentration across the data lines from 1 μg/mL to 1 fg/mL.

FIG. 5C illustrates control experiments with non-specific drug metabolites using norfentanyl ab-Au-sc-SWCNT devices, wherein the order of the drug metabolites bars is the same at each concentration.

FIG. 6A illustrates FET characteristic curves for norfentanyl sensing via a direct coupling approach in 1000× diluted synthetic urine using norfentanyl ab-sc-SWCNT devices and the direction of the arrow represents the increasing concentration across the data lines from 1 ag/mL to 1 μg/mL.

FIG. 6B illustrates a calibration plot of the devices for norfentanyl sensing in different dilutions of synthetic urine; and via an AuNP approach, wherein all data points plotted in the calibration plots are Mean±SD, and the number of devices (n) used for calculation are indicated in the parenthesis in the legend.

FIG. 6C illustrates FET characteristic curves for norfentanyl sensing in synthetic urine without dilution using norfentanyl ab-Au-sc-SWCNT devices, and the direction of the arrow represents decreasing concentration across the data lines from 1 μg/mL to 1 ag/mL.

FIG. 6D illustrates calibration plot of the devices for norfentanyl sensing in different dilutions of synthetic urine, wherein all data points plotted in the calibration plots are Mean f SD, and the number of devices (n) used for calculation are indicated in the parenthesis in the legend.

FIG. 7A illustrates schematically a flexible gold FET for norfentanyl sensing with coplanar gate (AUFET), wherein the zoom-in view is an illustration of interdigitated electrodes.

FIG. 7B illustrates norfentanyl sensing using AUFET by functionalizing the sc-SWCNTs with norfentanyl antibody via the direct coupling approach, and the direction of the arrow in the inset represents increasing concentration across the data lines from 1 ag/mL to 1 μg/mL.

FIG. 7C illustrates norfentanyl sensing using AUFET by attaching the norfentanyl antibodies on the Au gate (Norfentanyl ab@Au gate) and the direction of the arrow in the inset represents increasing concentration across the data lines from 1 ag/mL to 1 μg/mL.

FIG. 7D illustrates norfentanyl sensing in synthetic urine using norfentanyl ab@Au gate AUFET.

FIG. 7E illustrates a comparison of norfentanyl sensing performances between whole antibody and reduced antibody functionalized AUFET.

FIG. 8 illustrates a calibration plot of fentanyl sensing using reduced fentanyl antibody (r-fentanyl-ab)-functionalized Au-SWCNT FET sensors.

FIG. 9A illustrates a perspective view of an embodiment of a handheld sensor system hereof.

FIG. 9B illustrates a perspective view of an embodiment of the handheld sensor system of FIG. 9A hereof wherein a sensor module is removed from the system.

FIG. 9C illustrates a perspective, cutaway view of the handheld sensor system of FIG. 9A.

FIG. 9D illustrates schematically an embodiment of a voltage divider configuration of resistors for use in the electronic circuitry of the system of FIG. 9A, where a change in resistance is converted to a change in voltage.

FIG. 9E illustrates schematically an embodiment of a Wheatstone bridge for use in the electronic circuitry of the system of FIG. 9A.

FIG. 9F illustrates schematically an embodiment of electronic circuitry for use in connection with the system of FIG. 9A.

### DETAILED DESCRIPTION

It will be readily understood that the components of the embodiments, as generally described and illustrated in the figures herein, may be arranged and designed in a wide variety of different configurations in addition to the described representative embodiments. Thus, the following more detailed description of the representative embodiments, as illustrated in the figures, is not intended to limit the scope of the embodiments, as claimed, but is merely illustrative of representative embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “a,” “an”, and “the” include plural references unless the context clearly dictates otherwise. Thus, for example, reference to “an antibody fragment” includes a plurality of such antibody fragments and equivalents thereof known to those skilled in the art, and so forth, and reference to “the antibody fragment” is a reference to one or more such antibody fragments and equivalents thereof known to those skilled in the art, and so forth. Recitation of ranges of values herein are merely intended to serve as a shorthand method of referring individually to each separate value falling within the range. Unless otherwise indicated herein, each separate value, as well as intermediate ranges, are incorporated into the specification as if individually recited herein. All methods described herein can be performed in any suitable order unless otherwise indicated herein or otherwise clearly contraindicated by the text.

The terms “electronic circuitry”, “circuitry” or “circuit,” as used herein include, but are not limited to, hardware, firmware, software, or combinations of each to perform a function(s) or an action(s). For example, based on a desired feature or need, a circuit may include a software controlled microprocessor, discrete logic such as an application specific integrated circuit (ASIC), or other programmed logic device. A circuit may also be fully embodied as software. As used herein, “circuit” is considered synonymous with “logic.” The term “logic”, as used herein includes, but is not limited to, hardware, firmware, software, or combinations of each to perform a function(s) or an action(s), or to cause a function or action from another component. For example, based on a desired application or need, logic may include a software controlled microprocessor, discrete logic such as an application specific integrated circuit (ASIC), or other programmed logic device. Logic may also be fully embodied as software.

The term “processor,” as used herein includes, but is not limited to, one or more of virtually any number of processor systems or stand-alone processors, such as microprocessors, microcontrollers, central processing units (CPUs), and digital signal processors (DSPs), in any combination. The processor may be associated with various other circuits that support operation of the processor, such as random access memory (RAM), read-only memory (ROM), programmable read-only memory (PROM), erasable programmable read only memory (EPROM), clocks, decoders, memory controllers, or interrupt controllers, etc. These support circuits may be internal or external to the processor or its associated electronic packaging. The support circuits are in operative communication with the processor. The support circuits are not necessarily shown separate from the processor in block diagrams or other drawings.

The term “controller,” as used herein includes, but is not limited to, any circuit or device that coordinates and controls the operation of one or more input and/or output devices. A controller may, for example, include a device having one or more processors, microprocessors, or central processing units capable of being programmed to perform functions.

The term “software,” as used herein includes, but is not limited to, one or more computer readable or executable instructions that cause a computer or other electronic device to perform functions, actions, or behave in a desired manner. The instructions may be embodied in various forms such as routines, algorithms, modules, or programs including separate applications or code from dynamically linked libraries. Software may also be implemented in various forms such as a stand-alone program, a function call, a servlet, an applet, instructions stored in a memory, part of an operating system or other type of executable instructions. It will be appreciated by one of ordinary skill in the art that the form of software is dependent on, for example, requirements of a desired application, the environment it runs on, or the desires of a designer/programmer or the like.

As used herein, an “antigen” (sometimes abbreviated Ag) is any substance that can be specifically recognized by an antibody. Certain antigens induce a body to make an immune response against the antigen. Antigens may include toxins, chemicals, viruses, bacteria, and other substances having an origin outside the body. Antigens may include a molecule or molecular structure which, for example, may be present on the outside of a pathogen. Antigens may also have an origin within the body. Body tissues and cells (for example, cancer cells) may also include antigens that cause an immune response. Further, hormones may be antigens. Antigens can be bound by antigen-specific antibodies or by a B-cell antigen receptor. Antigens may be proteins, peptides (which are chains of amino acids) or polysaccharides (which are chains of monosaccharides or simple sugars). Lipids and nucleic acids may become antigens when combined with proteins and polysaccharides. An “antibody” (sometimes abbreviated as Ab and sometimes referred to as an immunoglobulin or Ig), is a relatively large, Y-shaped protein that is used by the immune system to identify and neutralize foreign objects (including pathogenic bacteria and viruses). As used herein, an “antigen receptor” is a protein which selectively binds/interacts with an antigen.

The antigen analyte may, for example, be an antigen of a pathogen selected from the group of a viral pathogen and a bacterial pathogen. The pathogen may, for example, be SARS-CoV-2, HIV, tuberculosis, syphilis, hepatitis (for example, hepatitis b or c), E. coli, Salmonella, Pseudomonas aeruginosa, Influenza, Staphylococcus aureus, or Streptococcus pyogenes, cytomegalovirus (CMV), or Epstein-Barr virus (EBV). In a number of embodiments, the pathogen is SARS-CoV-2. The antigen may, for example, be a spike antigen (SAg) or a nucleocapsid protein antigen (NAg) of SARS-CoV-2 and the antibody is anti-SARS-CoV-2 spike protein antibody (SAb) or anti-SARS-CoV-2 nucleocapsid protein antibody. In a number of embodiments, the antigen is a spike antigen (SAg) of SARS-CoV-2 and the antibody in the anti-SARS-CoV-2 spike protein antibody.

Many other antigens, including non-pathogenic antigens may be detected by the sensors hereof. Such antigens include, for example, substances originating within the body (for example, hormones such as cortisol) and substances originating outside of the body. Antigens originating outside the body (and metabolites thereof within the body) may, for example, include drug molecules or drug metabolites. Representative example of drugs include fentanyl, cocaine, morphine, hydrocodone, codeine and tetrahydrocannabinol (THC). Representative examples of drug metabolites include norfentanyl and 6-Acetylmorphine (6-AM).

In general, nanostructures are structures of intermediate size between microscopic and molecular structures. Nanostructures may, for example, have at least one dimension in the range of 0.1 to hundreds of nanometers. Many nanostructures have at least one dimension in the range of 1 to 100 nm. Nanotubes are, for example, considered two-dimensional nanostructures and may have a diameter in the range of, for example, 0.1 nm to hundreds of nm and a length that may be significantly greater than the diameter.

Chemically sensitive solid-state resistors (chemiresistors) and field effect transistors (FETs) hereof may, for example, exhibit room temperature liquid phase sensitivity to antigens (or antibodies) in, for example, a clinical sample such as a saliva, nasopharyngeal swabs, serum/plasma, bronchoalveolar lavage (BAL), or endotracheal aspirate (ETA). In general, any biological fluid/fluid sample may be analyzed to determine if the fluid sample includes an analyte above the detection level of a device hereof. In certain embodiments, it is also possible to analyze breath (for example, droplets of fluid from breath). In a nanostructure-based FET device, one, for example, measures electrical current through nanostructures such as sc-SWCNT under an applied gate voltage. In chemiresistor devices, a gate voltage is not applied. In both types of devices, an electrical property (for example, conductance or resistance) of nanostructures such as nanotubes changes upon exposure to an analyte, thereby providing a sensor signal. Depending on the semiconducting nature of the nanostructures, application of a gate voltage can provide amplification of the sensor signal. Nanotubes such as single-walled carbon nanotubes or SWCNTs and, particularly, sc-SWCNTs provide an ideal candidate for incorporation into extremely small and low power devices hereof because they demonstrate extreme environmental sensitivity, high electrical conductivity, and inherent compatibility with existing microelectronic fabrication techniques.

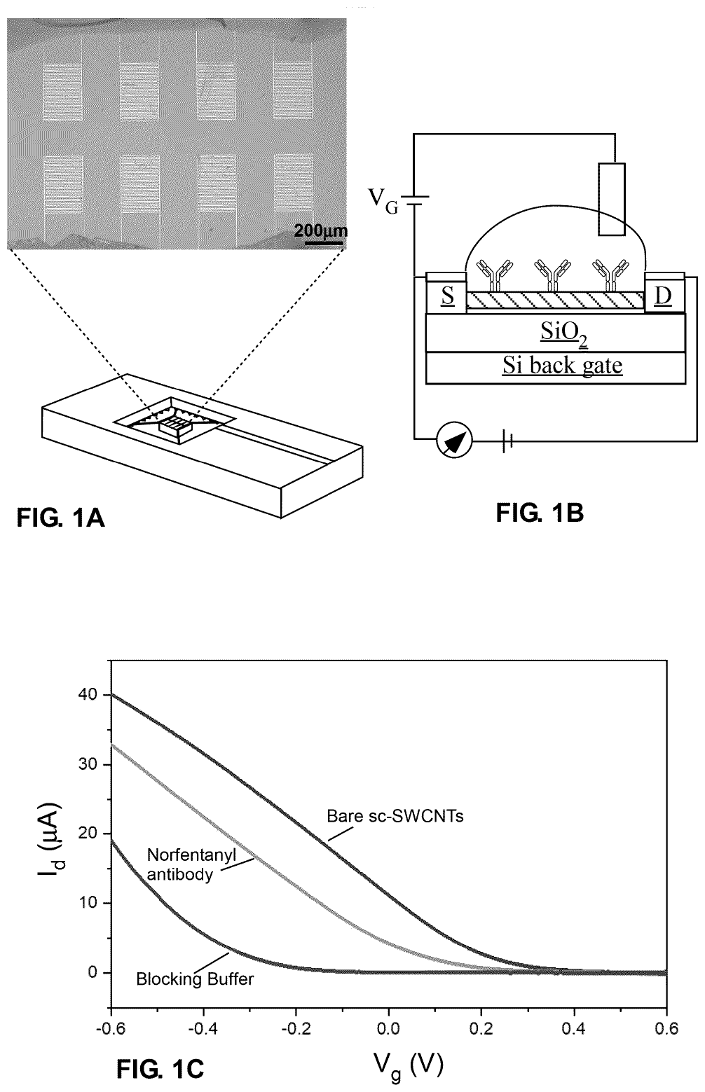

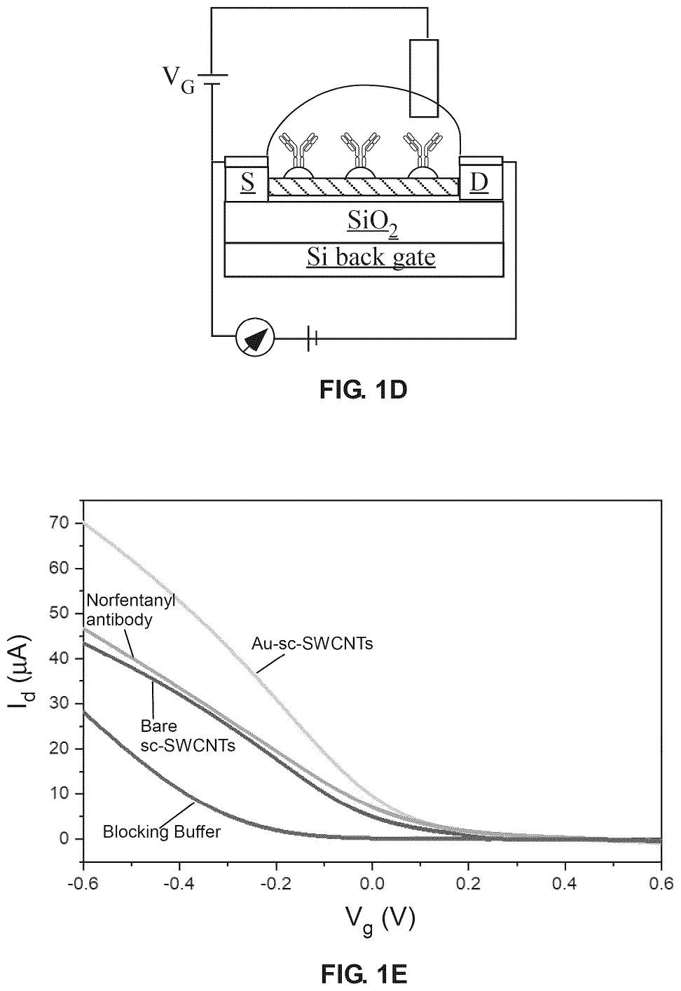

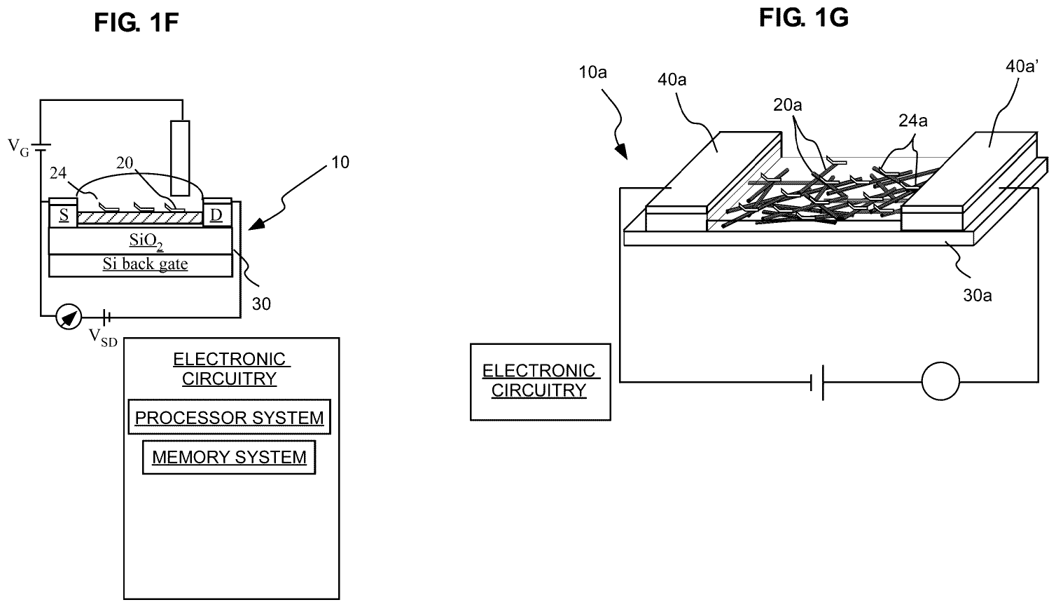

The upper portion of FIG. 1A provides a top optical image of an embodiment of a sensing chip with 8 devices in an upper portion thereof. The lower portion of FIG. 1A illustrates schematically an embodiment of a sensing chip that was wire-bonded in a package for measurements. FIG. 1B is a schematic illustration of a norfentanyl-antibody-functionalized SWCNT-based FET biosensor formed via direct coupling of the antibody to the SWCNT as described further below. FIG. 1C illustrates FET transfer characteristics of each functionalization step using the direct coupling approach. FIG. 1D illustrates schematically an embodiment of norfentanyl antibody functionalized SWCNT-based FET biosensor formed via an AuNP coupling approach described further below. FIG. 1E illustrates FET transfer characteristics of each functionalization step using AuNP approach.

A schematic representation of embodiment of an FET sensor device 10 hereof is set forth in FIG. 1F, while an embodiment of a chemiresistor sensor device 10a hereof is illustrated schematically in FIG. 1G. The illustrated sensor devices 10, 10a include a sensing medium material including one or more representative nanostructures. Such nanostructures include, for example, sc-SWCNTs 20,20a. In a number of embodiments, the nanostructures are a network of sc-SWCNTs). Single walled carbon nanotubes are classified based on their electrical properties. Nanotubes may, for example, be considered to be either semiconducting or metallic. The nanotube synthesis process typically yields a mix of both metallic and semiconducting nanotubes. Purification steps are required to enrich the samples to be either mostly metallic or mostly semiconducting. Either mixed or purified nanostructures may be used in the sensor systems hereof. However, purified semiconducting nanostructures may provide improved, lower levels of detection and a wider dynamic range in devices, systems, and methods hereof. As used herein, the term “semiconductor enriched” (in reference to nanostructures such as sc-SWCNTs) indicates that there is a semiconducting content of at least 66%. In a number of embodiments, the semiconducting content is at least 90%, at least 95%, at least 99%, or at least 99.9%. In general, a greater semiconducting content will result in a better output signal.

In single-walled carbon nanotubes, all carbon atoms are located on the surface where current flows, making a stable conduction channel that is extremely sensitive to a surrounding chemical environment. Nanotubes and other nanostructures, including single walled nanotubes (SWCNTs) such as SWCNT's, have the ability to change conductance in response to interaction with analytes. This characteristic is, for example, implemented in a number of embodiments of systems 10 and 10a (see FIGS. 1F and 1G).

Various nanostructures other than SWCNTs are suitable for use herein. Such nanostructures include, but are not limited to, multi-walled carbon nanotubes, graphene nanosheets and their derivatives (for example, reduced graphene oxide and holey graphene), nanowires, nanofibers, nanorods, nanospheres, nanoribbons (for example, interconnected nanoribbons of holey reduced graphene oxide) or the like, or mixtures of such nanostructures. Moreover, in addition to carbon, those skilled in the art will appreciate that the nanostructures hereof can be formed of boron, boron nitride, and carbon boron nitride, silicon, germanium, gallium nitride, zinc oxide, indium phosphide, molybdenum disulfide, silver, and/or other suitable materials. The formation and/or function of reduced graphene oxide and holey graphene compositions are, for example, discussed in U.S. Pat. Nos. 8,920,764, 9,482,638, and 10,801,982, and U.S. Patent Application Publication No. 2021/0122638, the disclosures of which are incorporated herein by reference.

As illustrated in FIGS. 1F and 1G, the sensing medium or material, including semiconducting sc-SWCNTs or a network of sc-SWCNTs 20 (or other nanostructures), may, for example, be disposed upon a substrate 30 (for example, silicon dioxide or quartz) and contacted by two conductive (for example, metallic—such as Au and/or Ti) electrodes representing a source (S) (a conductive electrode or terminal) and a drain (D) (a conductive electrode or terminal). In the operation of an FET circuit such as illustrated in FIG. 1F, changes in electrical conductivity may, for example, be measured for an applied gate voltage. One may, for example, measure current flow between source (S) and drain (D) as a function of a swept/varied gate voltage range. In the liquid phase, the sensing material may, for example, be covered in liquid.

As described above, a chemiresistor sensor device such as device 10a need not include an applied gate voltage. In chemiresistor 10a the sensing medium or material, including nanostructures 20a, bridges the gap between two conductive electrodes 40a and 40a′ (for example, gold electrodes), which may be referred to a source and a drain. The sensing medium or material may alternatively be immobilized upon a set of interdigitated electrodes. The resistance/conductance between electrodes 40a and 40a′ can be readily measured. The sensing medium or material has an inherent resistance/conductance that is changed by the presence of the analyte. In a chemiresistor, a source-drain bias voltage may, for example, be swept through a range of voltages, and drain current may be measured.

As described above, in a number of embodiments, the sensing media or materials hereof include one or more antibody fragments such as reduced antibodies immobilized upon the nanostructures thereof—for example, via covalent attachment of the antibody(ies), antigen receptor(s), or antigen(s) to the nanostructure, via a gold-thiol interaction/bonding, etc. In a number of embodiments, reduced antibodies 24, 24a are covalently attached to nanostructures 20, 20a to provide detection of antigens or attached, for example, via gold-thiol interaction/bonding. By immobilizing and antibody or antibody fragment/reduced antibody on the nanostructures in such a manner, more robust sensors may be achieved compared to various other immobilization techniques. One may, for example, readily remove (for example, via simple rinsing or washing) unbound molecular species. The immobilization methodologies hereof allow the achievement of increased sensitivities.

Problems in reproducibility may result from uncontrollable deposition and functionalization of nanostructures such as sc-SWCNT on the devices hereof, which may result in variance between devices. Significant device-to-device variability may, for example, make the calibration of devices more difficult, resulting in a requirement to calibrate each device individually. To improve reliability and standardized detection, controllable deposition and alignment of nanostructure/SWCNT flakes on devices hereof may be important in certain embodiments. For example, uniform SWCNT networks can be formed in the channel between electrode by inkjet printing or dip coating (for example, for fabricating thin-film transistors or TFTs) with <10% variability.

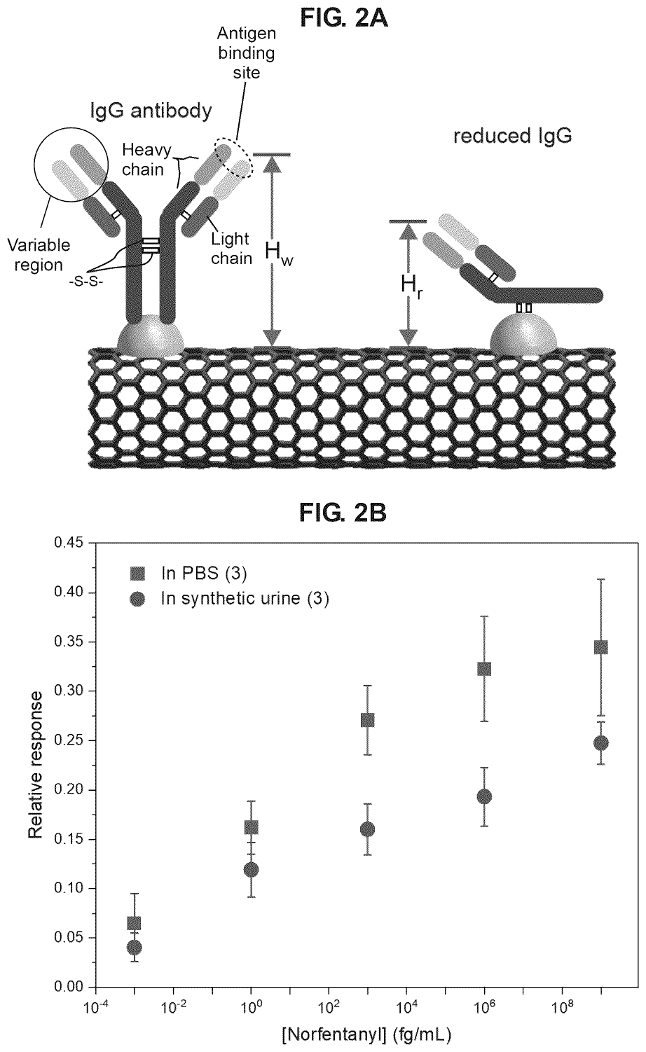

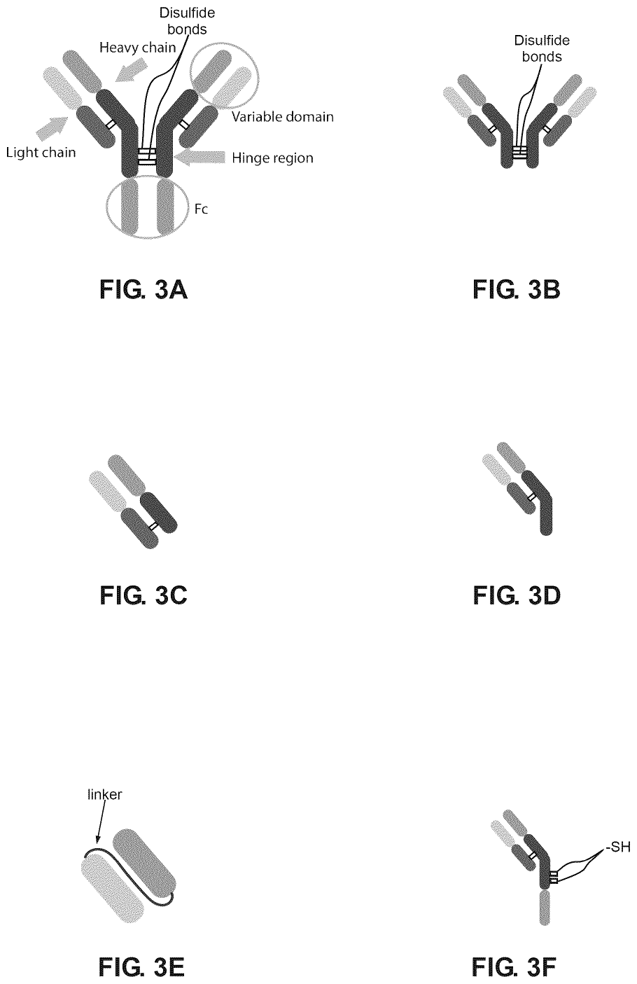

In a number of embodiments, sensor devices hereof are functional for detection of an antigen in a fluid sample. As described above, the sensor medium includes a plurality of nanostructures (for example, SWCNT having an enriched semiconducting content) and one or more antibody fragments immobilized on the plurality of nanostructures. As, for example, illustrated in FIGS. 2A and 3A, the structure of antibodies (which are proteins) includes two pairs of polypeptide chains (lengths of amino acids linked by peptide bonds) that form a flexible Y shape. The stem or lower portion of the Y includes one end of each of two identical heavy chains, while each arm or upper portion of the Y is composed of the remaining portion of a heavy chain plus a smaller protein referred to as the light chain. The two light chains are also identical. The two heavy chains are linked to each other by disulfide (—S—S—) bonds. Each heavy chain is linked to a light chain by a disulfide bond. The ends or tips of each arm of the Y forms a binding site that binds antigen. Thus each antibody has two identical antigen-binding sites, one at the end of each arm. The antigen-binding sites vary greatly among antibodies. See FIGS. 2A and 3A.

The sensitivity of FET biosensors may be limited by the Debye screening effect. To improve the sensitivity of biosensors hereof without compromising the antigen-antibody binding site interaction, a novel strategy hereof is to reduce the size of the biorecognition elements. In that regard, in a number of embodiments hereof antigen fragments are immobilized on the nanostructures hereof. Antigen-binding fragments that can be generated to include the variable regions of antibodies such as IgG include the F(ab′)2, Fab, Fab′, Fv and reduced antibody or “r-IgG” antigen-binding fragments. Fc fragments are generated from the heavy chain constant region of IgG antibodies, and therefore they do not contain the antigen-binding sites. These antigen-binding fragments vary in size (molecular weight), valency and heavy chain content. Various antibody fragments which includes active binding sites are, for example, illustrated in FIGS. 3B through 3F.

Referring to FIG. 3B, the F(ab′)2 (˜10 kDa) fragment includes two antigen-binding sites joined at the hinge through disulfides. F(ab′)2 fragments may, for example, be generated by cleaving the IgG antibody with pepsin, which separates the Fab fragments from the Fc fragment. FIG. 3C illustrates the Fab (˜50 kDa) fragment, which is a monovalent fragment. The Fab fragment includes a light chain and a portion of the heavy chain. The Fab fragment includes the entire antigen-binding region, including the variable domains of both heavy and light chains, but lacks the Fc fragment and the hinge region. FIG. 31) illustrates the Fab′ (˜55 kDa) fragment, which includes the variable domains of the heavy and light chains of the antibody, as well as a portion of the hinge region. The Fab′ fragment may also contain a portion (typically a small portion) of Fc. Fab′ fragments may, for example, be produced by proteolytic cleavage of the IgG antibody molecule with papain, or reduction of F(ab′)2 fragments. FIG. 3E illustrates the Fv (˜25 k Da), which is the smallest fragment produced from IgG that contains a complete antigen-binding site. Fv fragments may be unstable because the variable domains of the heavy and light chains are held together by non-covalent interactions. As illustrated in FIG. 3E, the cFv (single-chain Fv) has a flexible linker linking the variable domains of the heavy and light chains together, making the fragment more stable. FIG. 3F illustrates the reduced IgG (“r-IgG”) antibody fragment, which may be produced by using a mild reducing reagent that can selectively cleave the disulfide bridges in the hinge region of an IgG antibody. Although several disulfide bonds occur in IgG, those in the hinge-region are most accessible and easiest to reduce, especially with mild reducing agents such as 2-mercaptoethylamine (2-MEA). The product is a moiety with free thiol groups that can readily be used for antibody immobilization or enzyme labeling.

Each reduced antibody includes one side of the non-reduced, full or whole antibody (that is, one of the two sides of the Y shaped antibody). The one side of the Y-shaped antibody includes a heavy (polypeptide) chain, a light (polypeptide) chain attached to the heavy polypeptide chain, and an active binding site. See, for example, FIGS. 2A and 3F.

The sensor devices hereof further include electronic circuitry including at least one measurement system in operative connection with the sensor to measure a variable providing a measure of change in at least one property of the sensor medium which is dependent upon the presence of the antigen. Use of antibody fragment in the sensor media hereof provide, for example, significantly improved limits of detection compared to immobilization of the full antibody.

In representative studies hereof, semiconductor enriched (sc-) single-walled carbon nanotube (SWCNT)-based FET biosensors functionalized with reduced norfentanyl antibody for the sensitive detection of fentanyl or norfentanyl, the primary inactive metabolite of fentanyl, in, for example, urine samples were developed. Different sensor configurations were studied to achieve optimization of sensing results. Without limitation to any mechanism, by employing “reduced” antibodies, orientated immobilization of the norfentanyl antibody was achieved, bringing the antigen-antibody interaction closer to the sensor surface, which improved sensitivity. The norfentanyl biosensors hereof have a limit of detection in the fg/mL region in, for example, both calibration samples and synthetic urine samples, showing ultrasensitivity and high reliability.

Representative studies hereof demonstrate functionalized carbon nanotube-based biosensor for ultrasensitive norfentanyl detection in body fluids and, consequently, provide functionality for making time-sensitive decisions regarding fentanyl exposure. Representative norfentanyl, reduced antibody-functionalized sc-SWCNT-based FET biosensor achieved fg/mL level limits of detection of norfentanyl in both calibration samples and synthetic urine samples. To optimize the detection in the sensing matrix, different approaches for the attachment of antibody/reduced antibody on sc-SWCNTs were explored, namely the direct coupling approach and gold nanoparticle (AuNP) approach, and different biorecognition elements for the detection of norfentanyl. Distinct sensing behaviors were observed during the sensing experiments, implying different sensing mechanisms with different sensor configurations. By analyzing the sensing performances, it was found that the AuNP-decorated sc-SWCNT FET biosensors provide a more robust platform for antibody functionalization and are less susceptible to non-specific species present in biological samples, making them good candidate for point-of-care tool for the detection of fentanyl exposure. Moreover, the studies hereof demonstrated successful application of sensor fabrication with a flexible FET electrode and demonstrated good sensing performances for norfentanyl detection with a portable sensing setup.

Before discussing use of reduced antibodies as representative of antibody fragments, representative examples of the detection of fentanyl/norfentanyl are discussed wherein the full antibody is immobilized on a sensor medium including nanostructures for comparison. As shown in FIG. 1A, one embodiment of a sensor chip hereof contained eight devices with interdigitated gold source and drain electrodes and was packaged in a chip carrier for FET measurements. Once again, the FET channel length, (that is, interdigital gap) was 10 μm. Semiconductor-enriched (sc-) single-walled carbon nanotubes (SWCNTs) were deposited between interdigitated gold electrodes via dielectrophoresis (DEP), providing conducting channels as well as a platform for antibody/reduced antibody immobilization.

The attachment of antibody (or reduced antibody) on the sc-SWCNT FET devices was achieved through two different approaches: a direct coupling approach (FIG. 1B) and a gold nanoparticle (AuNP) approach (FIG. 1D). For direct coupling approach, norfentanyl antibodies were immobilized on sc-SWCNTs through covalent chemical bonds by EDC/NHS coupling between the carboxylic acid groups on the sidewalls of carbon nanotubes and the free amine groups on the antibodies. The AuNP approach utilized the surface of AuNPs decorated on the sc-SWCNTs for the binding of norfentanyl antibodies. For both types of FET devices, changes of the chemical environment of the sc-SWCNTs during each step of the functionalization process were reflected in the changes in the FET transfer characteristics. The direct coupling of norfentanyl antibodies induces a shift of threshold voltage toward more negative gate voltages and the decrease in the device conductance in the FET characteristics, and further shift of the threshold voltage and decrease in the conductance can be observed after the blocking buffer was applied (FIG. 1C). For Au-sc-SWCNT FET devices, while the decoration of AuNPs improves the conductivity of the sc-SWCNTs, yielding higher source-drain current in the p-type region, the attachment of antibodies on the AuNPs lowers the device conductance, and a similar negative shift of the threshold voltage and decrease in the conductance is observed in the I_{d}-V_{g} curve after the addition of the blocking buffer (FIG. 1E).

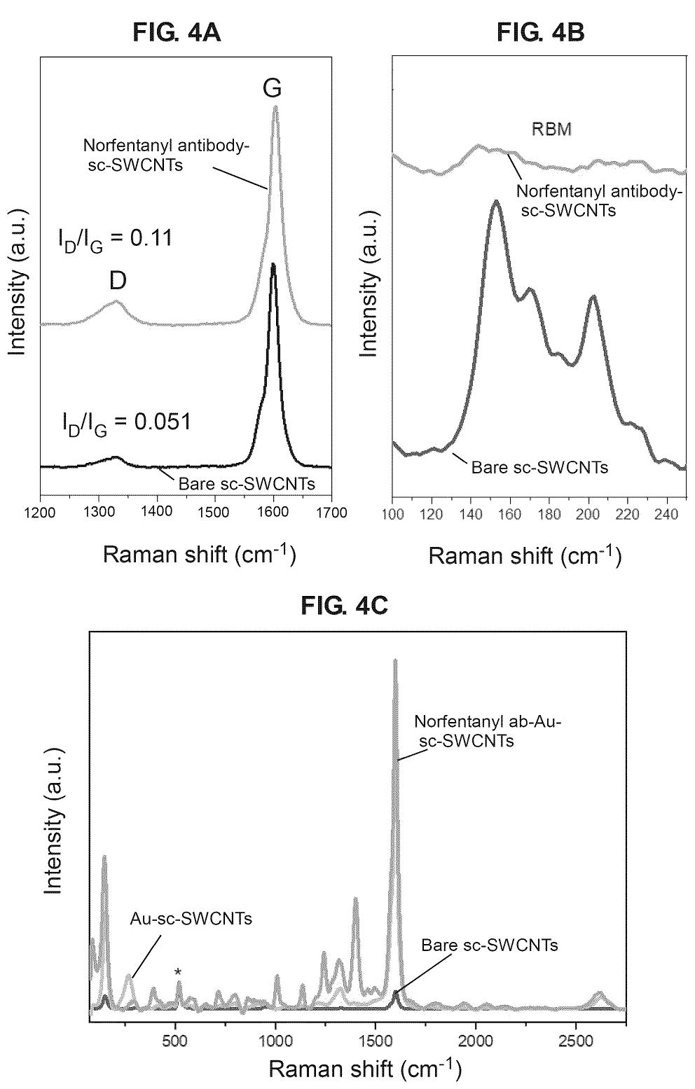

Scanning electron microscopy (SEM) images of the bare sc-SWCNT FET device showed the dense network of sc-SWCNTs. The covalent attachment of norfentanyl antibody was evidenced by the thickening of SWCNT strands with small nucleation of the antibody on the SWCNT surfaces. Additionally, the appearance of N 1 s peak in X-ray photoelectron spectroscopy confirmed the successful immobilization of antibodies on sc-SWCNTs. Raman spectroscopy was utilized to further understand the effect of antibody functionalization on the structure of SWCNTs. Although no prominent peaks from the antibody were observed after the direct coupling of the antibodies, the analysis of the D and G features from sc-SWCNTs reveal an increase in the I_{D}/I_{G} ratio from 0.051 to 0.11, suggesting an increase in the degree of functionalization of SWCNTs, likely due to the covalent bonding of antibodies to the SWCNTs (FIG. 4A). The intensity of the radial breathing mode (RBM) was also drastically reduced due to the functionalization on the side walls of sc-SWCNTs (FIG. 4B).

When introducing norfentanyl antibodies to AuNP-decorated SWCNTs, the antibodies predominantly bound to the AuNP surfaces as shown by AFM imaging. Average height of the AuNPs rose from 48.9±7.5 nm to 59.5±7.3 nm. The 10.6 nm increase in height matched the size of IgG type antibodies. The decoration of AuNPs on sc-SWCNTs also creates a substrate for surface enhanced Raman scattering (SERS). Raman intensity of sc-SWCNTs increased about 20 times due to the SERS effect. More importantly, Raman features from norfentanyl antibody, which are generally hard to resolve at low concentration, appear in the Raman spectra (FIG. 4C), indicative of the successful immobilization of antibodies on sc-SWCNTs.

While fentanyl, as a redox active molecule, can be detected using electrochemical methods such as cyclic voltammetry, DPV and SWV, no redox activity was found for norfentanyl when cyclic voltammetry studies were conducted with norfentanyl using sc-SWCNTs as the working electrode. The norfentanyl antibody-functionalized sc-SWCNT FET biosensor responses were investigated by employing a liquid-gate FET configuration and recording FET transfer characteristics (I_{d}-V_{g}), from which rich information about the biorecognition process, sensing mechanism, and sensing performances can be extracted. The FET transfer characteristics were measured using a portable dual-channel potentiostat to enable norfentanyl detection on site. Standard resistor tests suggest that no significant difference is observable between portable potentiostat and laboratory high precision sourcemeters. The latter were used in our previous SWCNT-based FET biosensor work.

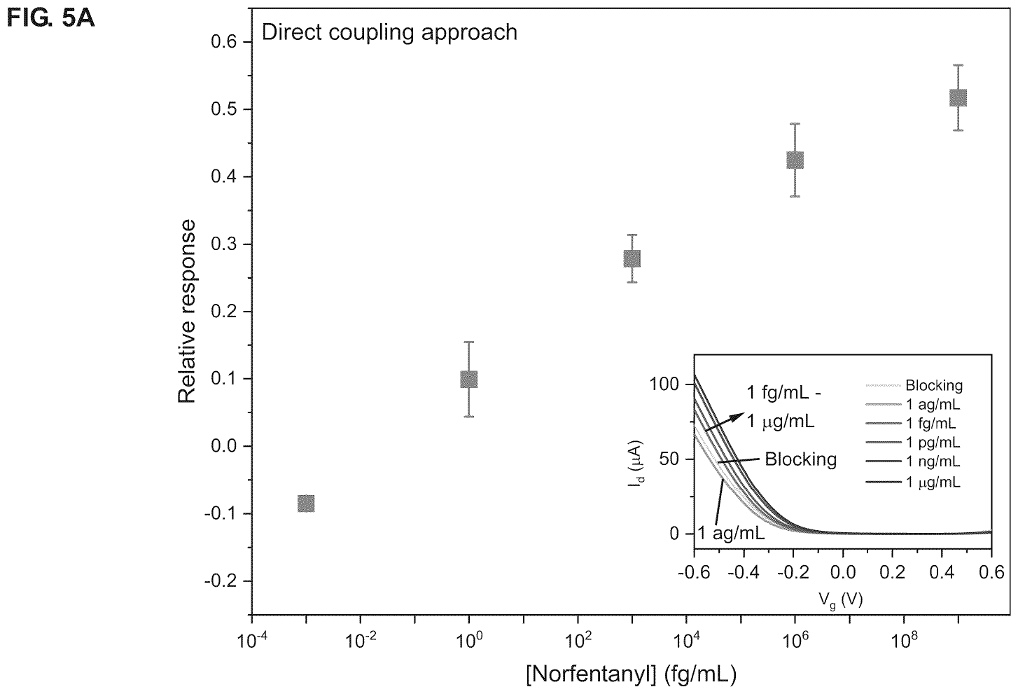

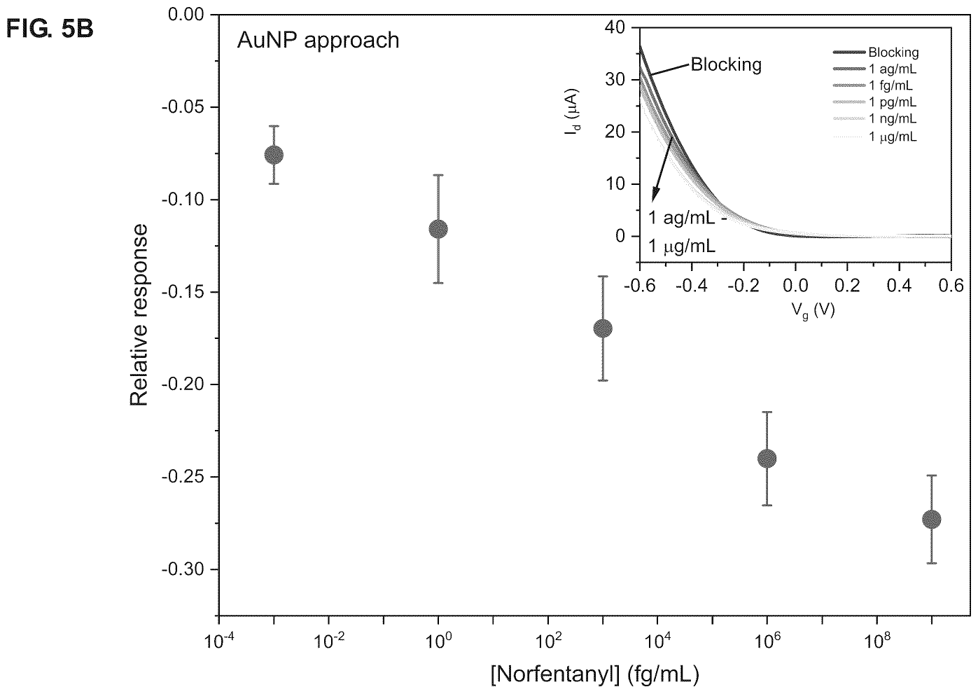

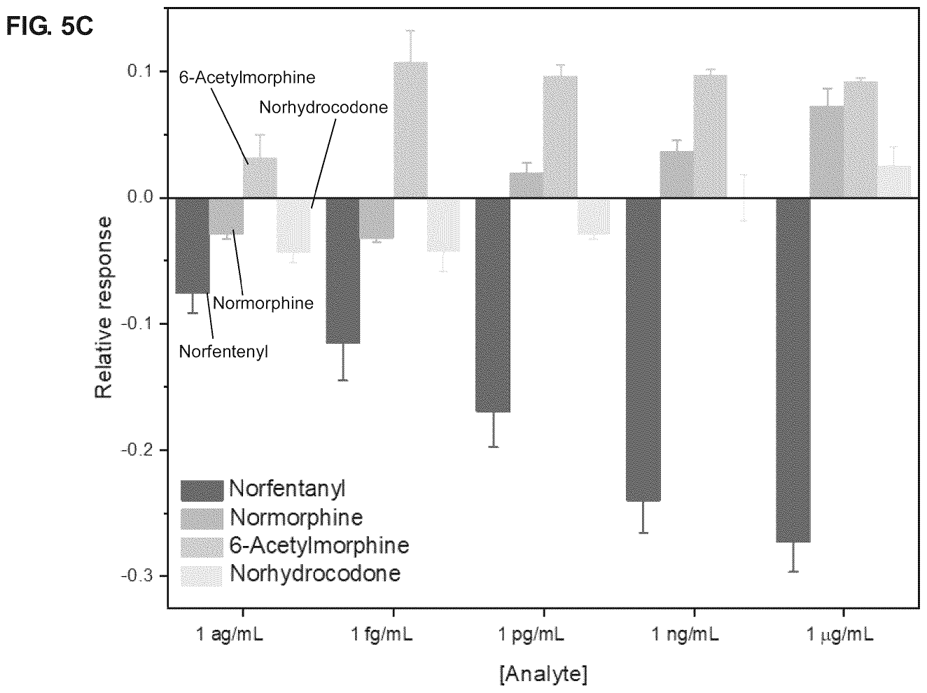

As shown in FIGS. 5A and 5B, both types of devices demonstrated sensing capabilities toward norfentanyl with limit of detection in the fg/mL region, but the calibration curve displayed opposite trends upon norfentanyl exposure. For devices adopting the direct coupling approach, the binding of norfentanyl on the device surface induced an increase in the conductance of the FET device, resulting in a corresponding increase in the relative response. This result is consistent with previously observed results with cortisol antibody-functionalized sc-SWCNT FET biosensors for cortisol sensing utilizing the same approach. Similarly, the binding between norfentanyl and norfentanyl antibody likely relies on the nonpolar hydrogen-π interaction and cationic-π interaction between norfentanyl and amino acid residues inside the binding sites of the antibody such as aspartate (Asp) and tyrosine (Try). Without limitation to any mechanism, the sensor response was attributed to the redistribution of charges on the antibody upon norfentanyl binding, making the antibody less positively charged and consequently p-doping the sc-SWCNTs.

When decorated with AuNPs, norfentanyl antibodies predominantly bound to the AuNP surfaces, thus the AuNP-nanotube interface is most responsible for the sensing. At the junction of metal nanoparticles and semiconductors, Schottky barriers form. Upon binding of norfentanyl, the charge redistribution of the norfentanyl antibody lowers the work function of the AuNPs, increasing the Schottky barrier, and as a result, a decrease in the conductance was observed in the experiment.

The specificity of both types of sensors were investigated using sc-SWCNT FET devices without the conjugation of norfentanyl antibodies. The lack of sensor responses when norfentanyl antibodies were absent indicated that the sensors have high specificity. In terms of selectivity, other opioid metabolites, namely normorphine (the metabolite of morphine), norhydrocodone (the metabolite of hydrocodone), and 6-acetylmorphine (indicative of heroin use), were added to the norfentanyl ab-Au-sc-SWCNT FET biosensors in the same concentration range as norfentanyl. However, the sensor behaviors are markedly different (FIG. 5C), further confirming that the observed sensor response with norfentanyl is associated with the specific interaction between norfentanyl and its antibody, allowing for good selectivity toward norfentanyl.

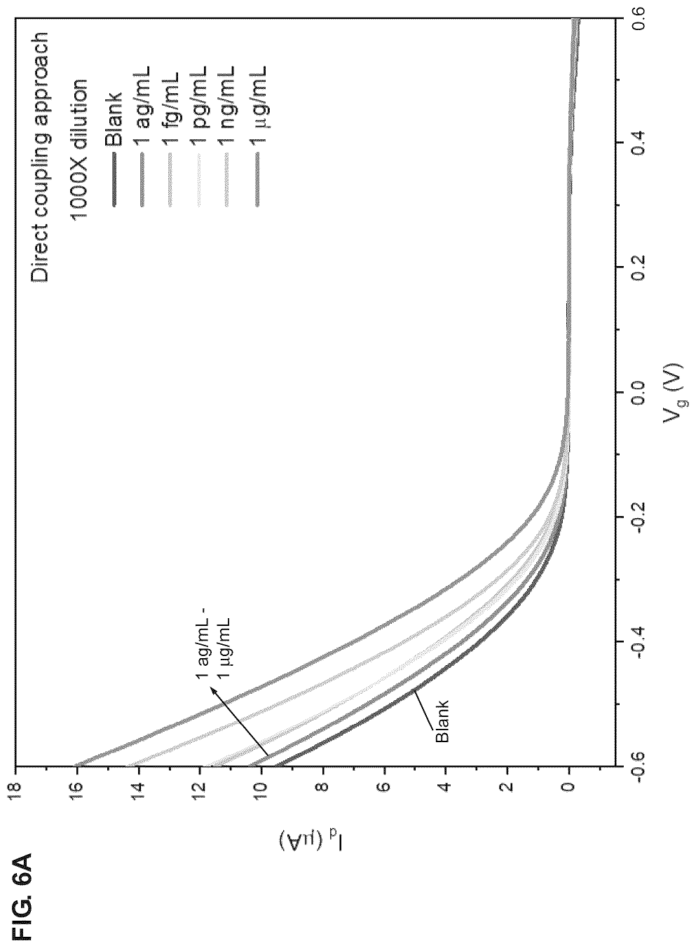

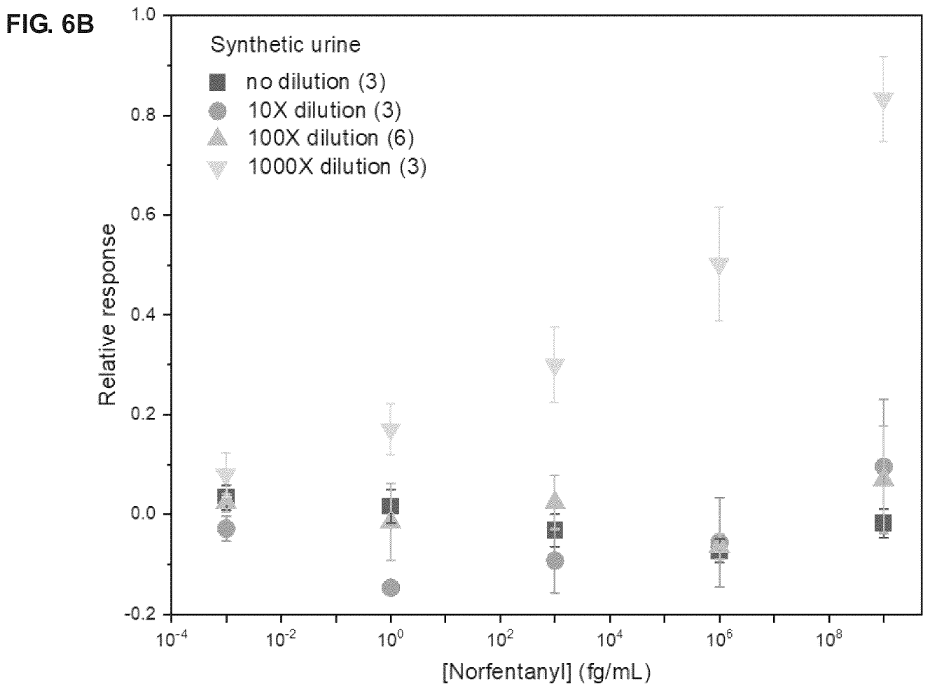

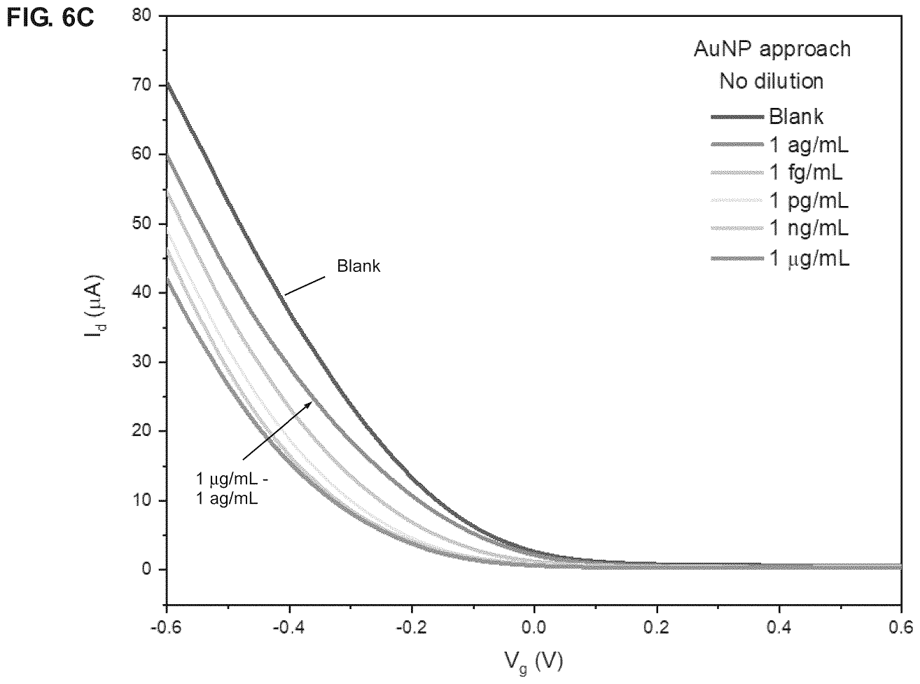

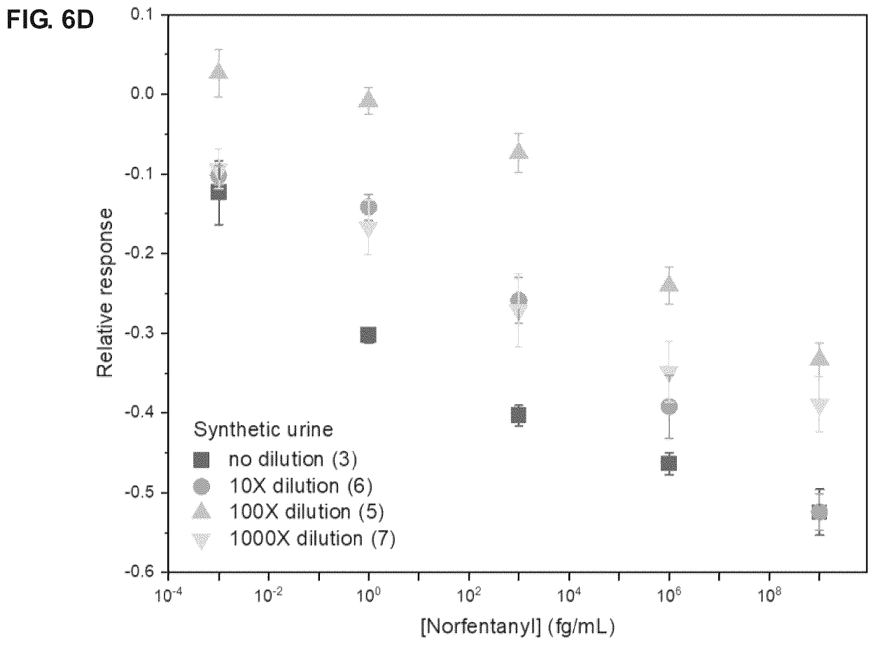

To study past fentanyl exposure of an individual, norfentanyl sensing experiments were conducted in synthetic urine (see Table I for synthetic urine composition) with both types of antibody-functionalized FET biosensors. The norfentanyl-containing synthetic urine samples were also diluted 10-, 100-, and 1000-fold in PBS to investigate the susceptibility of the sensors to interferences. For FET devices fabricated via the direct coupling approach, the sensitivities of the devices were drastically reduced in non-diluted, 10-fold and 100-fold diluted synthetic urine samples, and the norfentanyl sensing capability was observed only in 1000-fold diluted samples (FIGS. 6A and 6B). However, sensors with norfentanyl antibodies immobilized on AuNPs demonstrated similar norfentanyl sensing behavior regardless of the extent of dilution (FIGS. 6C and 6D).

TABLE 1

| Formula | Molarity (mM) | Quantity (g/100 mL) |
|---|---|---|
| CH₄N₂O | 249.750 | 1.5000 |
| KCl | 30.953 | 0.2308 |
| NaCl | 30.053 | 0.1756 |
| NH₄Cl | 23.667 | 0.1266 |
| NaH₂PO₄•2H₂O | 18.667 | 0.2912 |
| Na₂SO₄ | 11.965 | 0.1700 |
| C₄H₇N₃O | 7.791 | 0.0881 |
| Na₂HPO₄•2H₂O | 4.667 | 0.0831 |
| MgSO₄•7H₂O | 4.389 | 0.1082 |
| Na₃C₆H₅O₇•2H₂O | 2.450 | 0.0720 |
| CaCl₂ | 1.663 | 0.0185 |
| C₅H₄O₄N₃ | 1.487 | 0.0250 |
| K₂C₂O₄•H₂O | 0.19 | 0.0035 |

The compromised sensitivity of the sensors fabricated via the direct coupling approach may, for example, be due to the interactions between the non-specific species present in the synthetic urine with the carbon nanotubes. By immersing the FET devices in 1X synthetic urine without norfentanyl for 10 min, it is evident that the non-specific species in the synthetic urine can alter the FET characteristics of both types of devices. It is less likely that the antigen-antibody interaction is impaired because of two reasons: (1) the ionic strength of the synthetic urine is similar to that of PBS, which is optimal for antigen-antibody binding; and (2) while high concentration of urea (˜6 M) can be used for the dissociation of antigen-bound antibody, the urea concentration is relatively low (˜250 mM) in synthetic urine. With higher extent of dilution, the interactions between the interfering species and the carbon nanotubes became negligible, and the specific sensor responses restores. On the opposing side, for devices decorated with AuNPs, less carbon nanotube surfaces are exposed to the biological environment, therefore demonstrating less susceptibility to interferences.

Both types of sensors respond to the non-specific synthetic urine components in a similar way as to norfentanyl. As a result, a collective effect of the specific and non-specific interactions leads to an increase in the calibration sensitivity of both sensors in 1000-fold diluted synthetic urine. The comparison of the sensor performances in PBS and 1000-fold diluted synthetic urine reveals a 55.6% rise in calibration sensitivity for devices with directly coupled antibodies and a 55.2% rise in calibration sensitivity for devices decorated with AuNPs. However, to improve the quantitative ability of the sensors, in real-life practices, background elimination may be used when applying this biosensor for the detection of norfentanyl in clinical samples.

As described above, the sensitivity of FET biosensors is limited by the Debye screening effect, and sensor sensitivity of the representative norfentanyl FET biosensors (and other nanostructure-based biosensor for antigens) can be further improved, without compromising the antigen-antibody interaction, by reducing the size of the biorecognition elements. In a number of representative embodiments of use of an antibody fragments, a mild reducing reagent that cleaves the bridges (disulfide bridges as described above in the case of the norfentanyl antibody) in the hinge region of an IgG antibody was used to produce reduced antibodies with a free thiol group while leaving the binding site intact as described above. It was hypothesized that by employing the reduced antibody, the orientation of the antibody on the sc-SWCNT surface can be better controlled, and the antigen-antibody binding occurs closer to the sensor surface, thereby improving the sensitivity (see FIG. 2A). Although sensitivity was maintained using the direct route for attaching antibody fragments, the indirect route of attached in the antibody fragments to AuNPs immobilized upon the sc-SWCNTs provided significant improvement in sensitivity.

The binding between the representative reduced antibody and the AuNPs were analyzed by XPS. Peaks correspond to carbon (C), nitrogen (N), oxygen (O), gold (Au), and silicon (Si) can be found on the XPS survey spectrum, confirming the presence of the reduced antibodies on Au-sc-SWCNTs. Although no obvious sulfur (S) peak was shown in the survey spectrum, peaks associated with Au(I) appear in high-resolution XPS spectra of Au4f of the device. The peaks may be attributed to the covalent bonding between AuNPs and the free thiol terminals created by reducing the antibody, which confers the control over the orientation of the antibodies on the sensor surface. AFM characterization of the AuNP-sc-SWCNTs before and after the incorporation of reduced antibody revealed an average of 5.02 nm increase in the height of the nanoparticles, significantly lower than the 10.6 nm increase by attaching whole antibody. This result provides further evidence that by employing reduced antibodies, the antigen-antibody interaction can be brought closer to the sensing surface.

As expected, a calibration sensitivity obtained from the reduced antibody-functionalized sc-SWCNT FET devices was 0.039, which is 69.6% higher than devices functionalized with whole antibody. Without limitation to any mechanism, this result supports the hypothesis that the reduced distance between the binding site and the sensor surface mitigates the Debye screening effect and enhances the sensitivity of the sensor. An increasing trend in the relative response, however, was observed, which contrasts with the decreasing trend for whole antibody-functionalized AuNP-sc-SWCNT FET devices (FIG. 2B). Without limitation to any mechanism, one possible explanation for the opposite sensing behavior is that, by reducing the proximity of the binding sites to the AuNP surface, the binding between the norfentanyl, which relies mainly on the hydrogen-π interaction and cationic-π interaction, reduces the local electron density on the surface of AuNPs, thus increasing the work function of AuNPs, and consequently increase the conductance of the channels.

In non-diluted synthetic urine, the sensing capability of the reduced antibody-functionalized AuNP-sc-SWCNT FET biosensor for norfentanyl is preserved. A decrease in the calibration sensitivity of the devices is observed when compared to the sensitivity in PBS, which is due to the interferences in synthetic urine that induce negative relative responses of the devices.

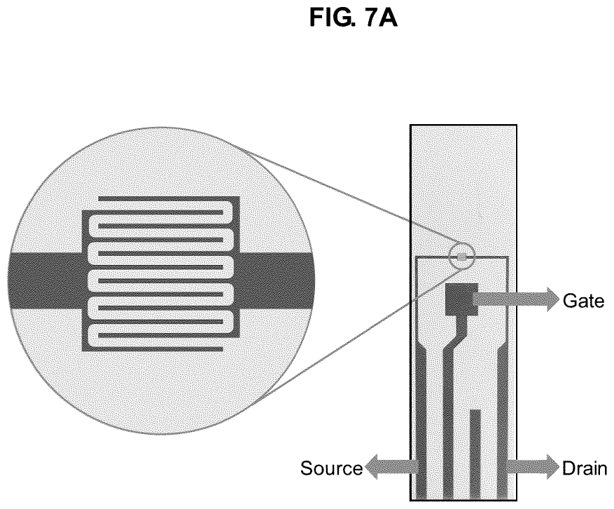

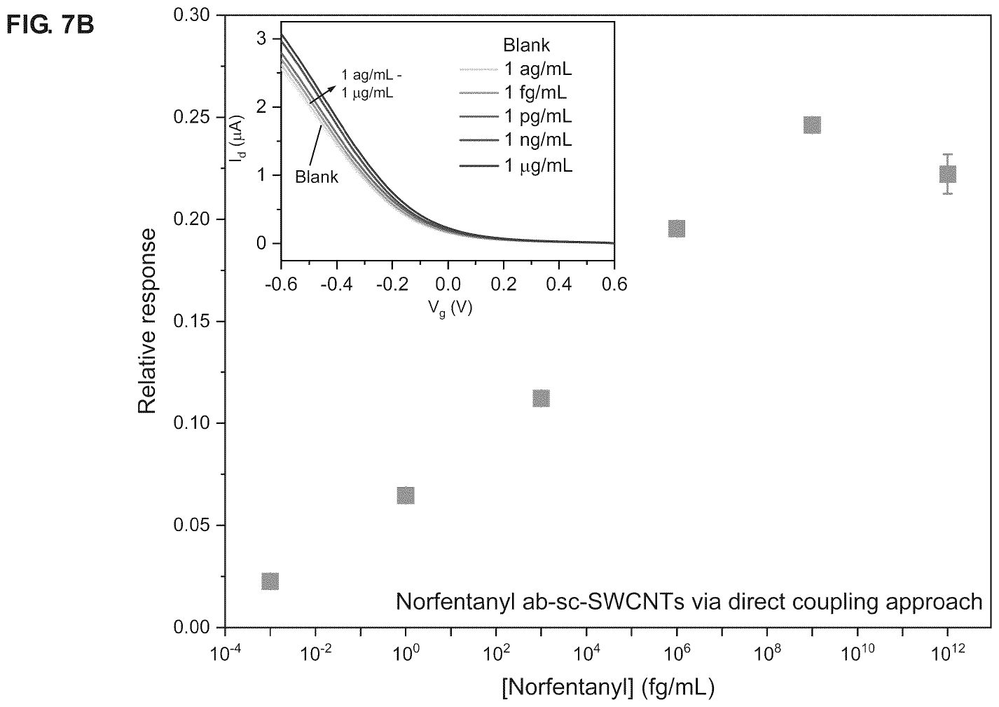

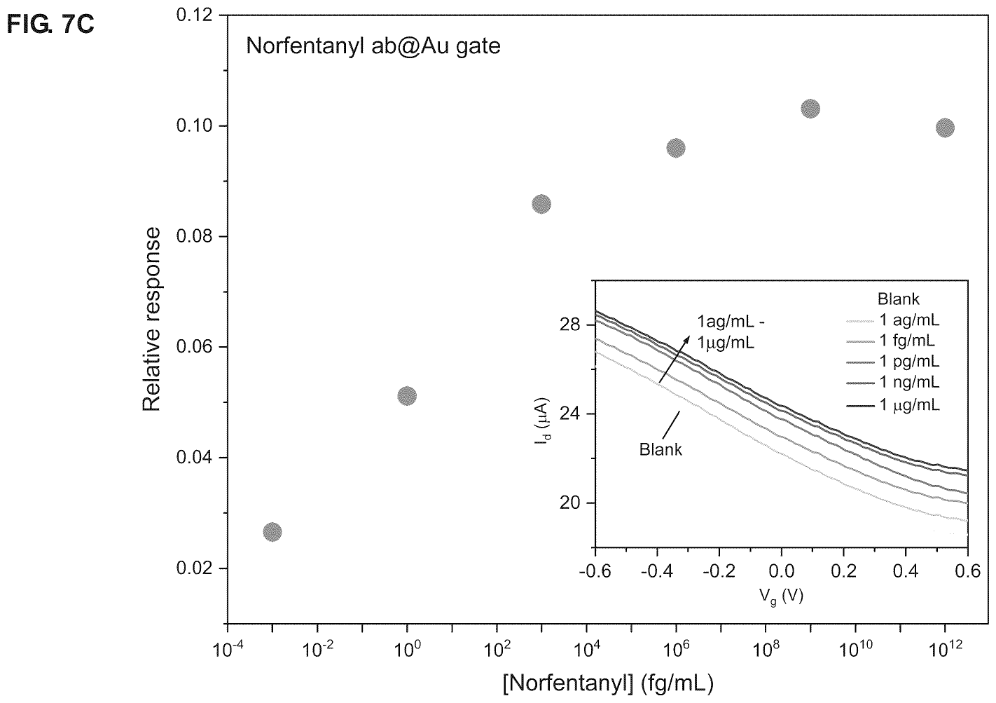

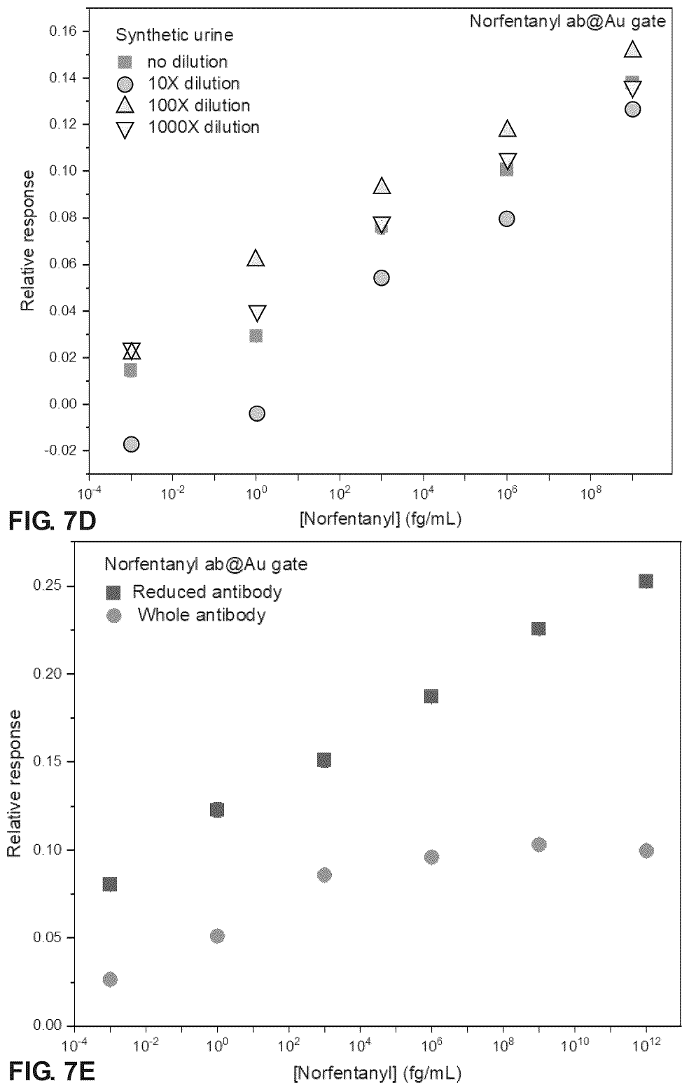

The sensor fabrication technique hereof was also applied on a commercially available flexible gold FET with coplanar gate (AUFET). In a number of studies a flexible gold field-effect transistors with coplanar gate, available from Metrohm of Riverview, Florida under produce number AUFET30 was used. The patterned gold gate on the AUFET eliminates the need for a separate gate electrode and fixes the distance between the gate electrode and the semiconducting channels, providing a more controlled sensor configuration (FIG. 7A). As a proof-of-concept, a similar sensor configuration was implemented on the AUFET as previously mentioned. Specifically, sc-SWCNTs were deposited in between the IDEs by drop-casting to ensure good conductivity, and norfentanyl antibody was first attached to the sensors via direct coupling. The sensing result in norfentanyl calibration samples exhibits similar sensing behavior with sensors fabricated on the Si chip and good sensing capability for norfentanyl (FIG. 7B). However, the small IDE area on the AUFET limits the deposition and functionalization of the sc-SWCNTs, resulting in lower reproducibility of the sensors.

Further studies took advantage of the gold gate for the immobilization of the biorecognition elements of the sensor. In that regard, by anchoring the antibodies on the gold gate, the binding between the analyte and the antibody alters the capacitance at the gate/electrolyte interface instead of direct modulation on the semiconducting channel. The gold gate, which was 3 mm×3 mm in size, provided a large surface area for spontaneous binding of norfentanyl antibodies. Upon addition of norfentanyl, the conductance of the device increased. The sensor calibration made by plotting the relative responses at applied gate voltage of −0.5 V against norfentanyl concentration validated the norfentanyl sensing effect of the AUFET sensor (FIG. 7c). Moreover, when tested in synthetic urine, the AUFET devices demonstrated consistent sensing performance regardless of the extent of dilution of synthetic urine, indicative of good reliability for quantitative detection of norfentanyl in complex matrix (FIG. 7D).

Switching the biorecognition element from whole antibody to reduced antibody further improves the sensitivity of the norfentanyl sensor (FIG. 7E). The smaller size of the reduced antibody, as well as the more controlled orientation on the gold surface, yields more available binding sites for norfentanyl on the sensing surface. The results suggest the great potential held by antibody-functionalized sc-SWCNT FET and other biosensors for the development of portable and reliable biosensors for detection of fentanyl exposure.

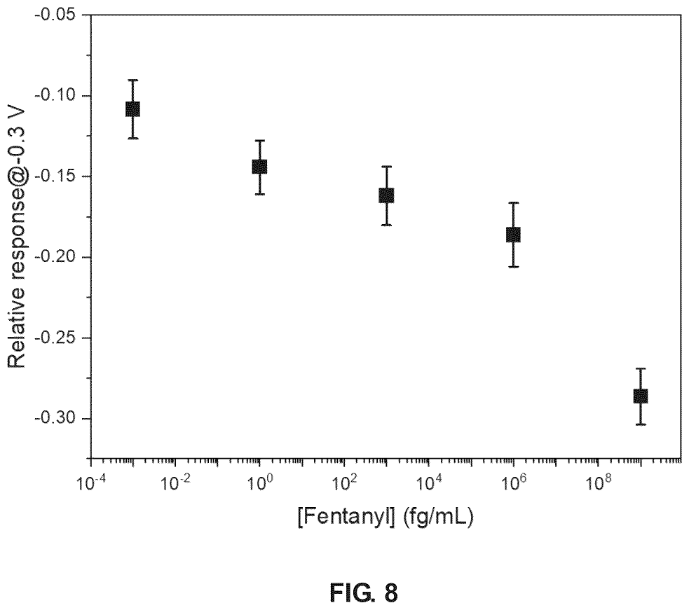

Studies of sensors hereof were also performed with reduced fentanyl antibody. Fentanyl antibody was purchased from commercial sources. In representative studies, reduced fentanyl antibody (r-fentanyl-ab) was attached to sc-SWCNT via a gold nanoparticle (AuNP) coupling approach as described above. Sensing experiments were performed using the same method described above for norfentanyl sensing. FIG. 8 illustrates fentanyl sensing results of an r-fentanyl-ab Au-SWCNT FET sensor in the form of a calibration plot. Unlike the case of norfentanyl sensing, the r-fentanyl-ab Au-SWCNT FET sensors hereof yielded a decreasing response upon the addition of fentanyl. With increasing concentration of fentanyl, a decreasing trend in the relative responses was observed, indicating the sensing capability of the r-fentanyl-ab Au-SWCNT FET sensors for fentanyl.

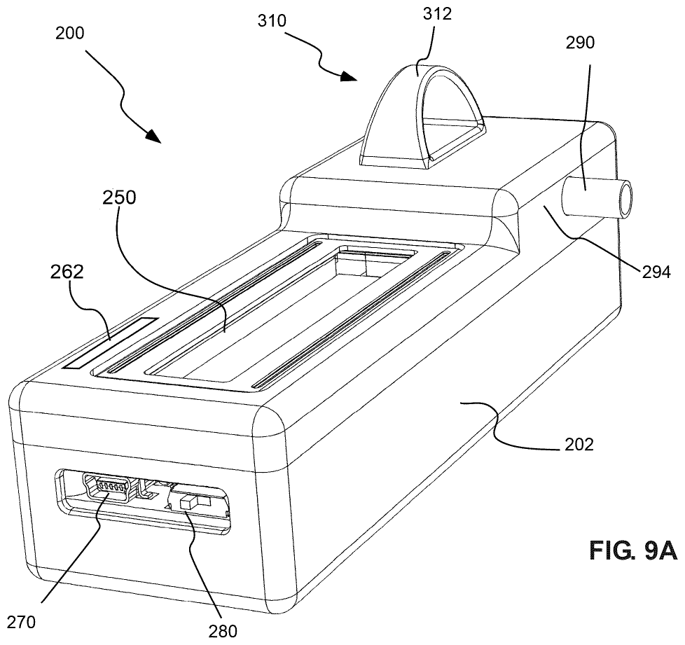

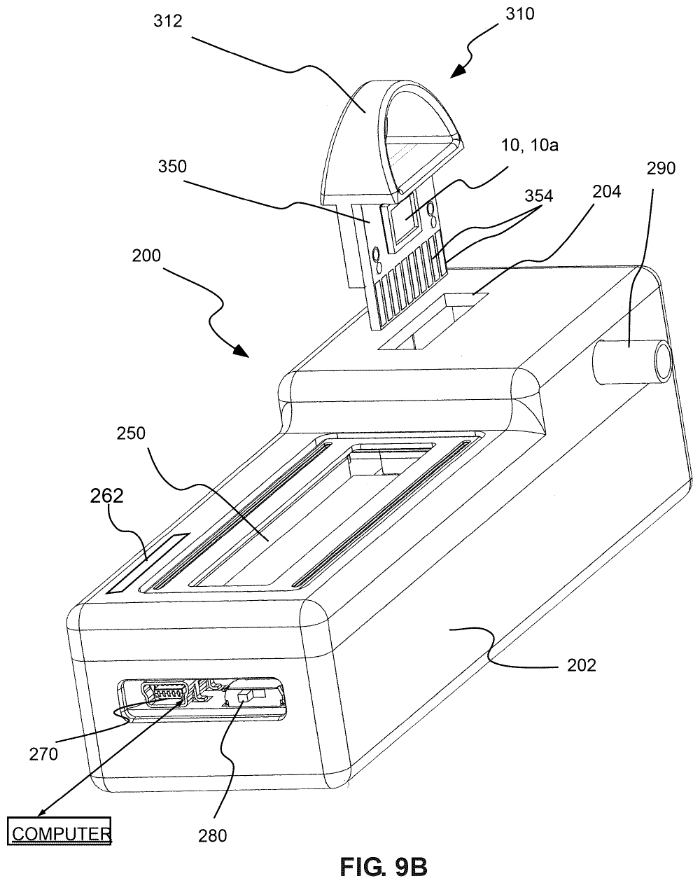

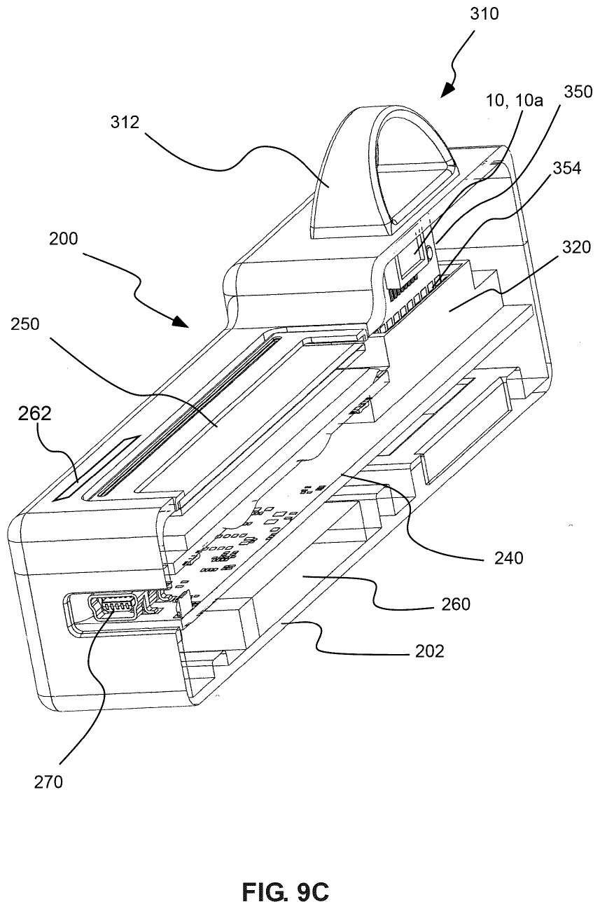

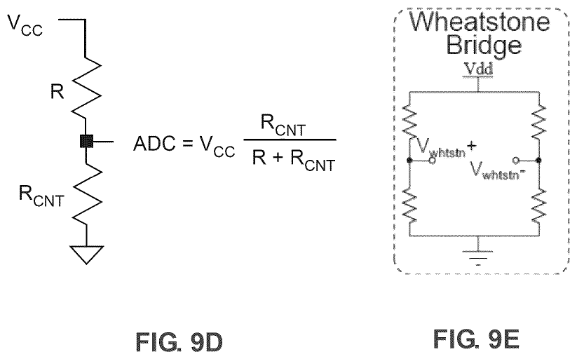

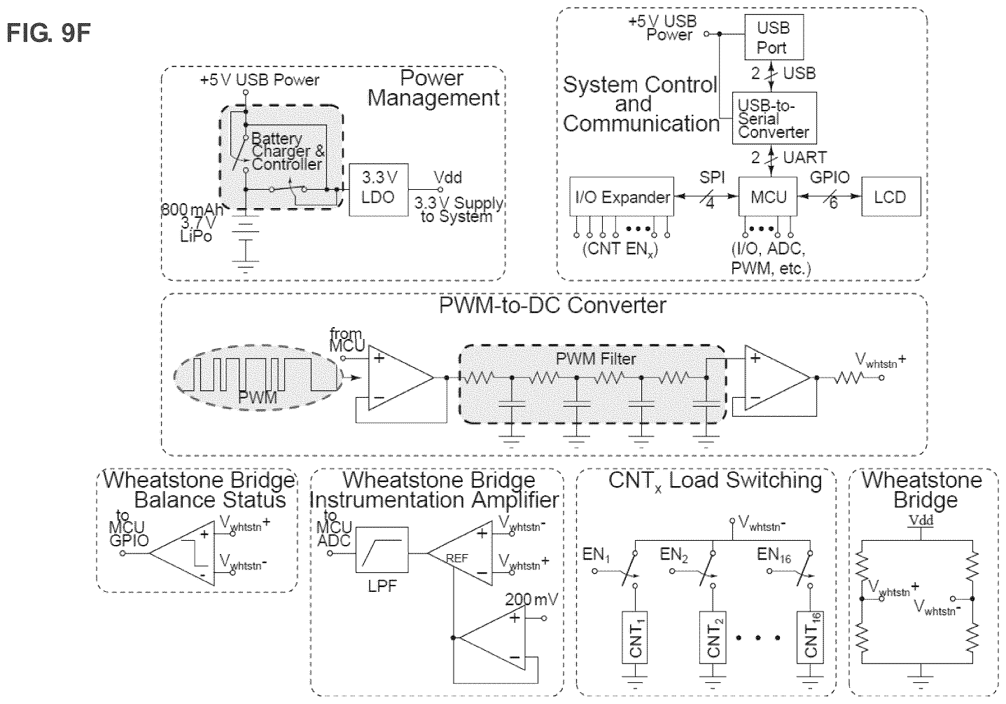

A sensor hereof may be incorporated as the sensing element of handheld sensor device or system 200 (as illustrated in FIGS. 9A through 9F). Such a system may, for example, include a processor system including a CC1110 chip (a system-on-chip including a microcontroller unit or MCU available from Texas Instruments of Dallas, Texas) to function as the microcontroller, a 3D printed case or housing (FIGS. 9A through 9C) to house the electronics (FIGS. 9A through 9F). The electronics illustrated in FIGS. 9D through 9F are for a chemiresistor and may include, for example, a Wheatstone Bridge to measure the resistance of the sensor. Alternatively, circuitry for FET sensors, as known in the art and represented by circuitry associated with the flexible gold field-effect transistor with coplanar gate described above, may be used in connection with handheld sensor devices or systems 200 hereof. A swappable and insertable sensor board (see, for example, FIGS. 9B and 9C) may, for example, be plugged in and removed from the system.

In the embodiment illustrated in FITS. 9A through 9C, device or system 200 hereof for detection of an antigen or an antibody provides a relatively compact form. Similar devices or systems are, for example, disclosed in PCT International Patent Application No. PCT/US2020/059591 and in U.S. Patent Publication Nos. 2020/0093429 and US2022/0365078, the disclosures of which are incorporated herein by reference. Device 200 includes a microchip-based sensor assembly 310 in which one or more chemiresistors 10 or field effect transistors (FETs) 10a is/are deposited on a silicon wafer 350 as illustrated in FIGS. 9B and 9C. Sensor assembly 310 is readily removably and operably attachable to a sensor assembly connector or receptacle 320 within housing 202 via a handle or gripping portion 312 on a first or outer end and conductive connectors 354 on a second or inner end. Sensor assembly 310 is placed in connection with a control board 240 (for example, a printed circuit board or PCB) via connector 220.

In the illustrated embodiment, sensor assembly 310 may be placed in and out of connection with connector or receptacle 320 via a slot or opening 204 formed in housing 202. The design of device 200 thereby facilitates removal of sensor assembly 310 for maintenance/replacement. A user may, for example, be provided with multiple sensor assemblies 310 in a system of kit for use in connection with device 200. Sensor assemblies 310, when removed from connection with device 200, may, for example, be serviced/refurbished or discarded.

Control board 240 of the electronic circuitry of device 200 includes or has attached thereto a controller system or processor system (not shown; including, for example, one or more microprocessors such as a CC1110 microprocessor) and a memory system (not shown) which is placed in operative or communication connection with the processor system via control board 240 or integrated with the processor system. Control board 240 may also be in operative connection with a display 250 such as a liquid crystal display. A power supply/battery 260 (for example, a Lithium Polymer or LiPo battery) may be supplied to power one or more electronic circuitry components as described above. Such electric circuitry components are housed within housing 202.

A mini USB or other communication port 270 in operative connection with control board 240 may extend through housing 202. Mini USB or other communication port 270 may, for example, be used to connect to a computer such as a general-purpose personal computer or PC (see, for example, FIG. 9B) to, for example, effect software revision and/or data transfer, to effect battery charging and/or to effect power the device (for example, even if battery 260 is absent or damaged) as known in the computer arts. An indicator 262 (for example, one or more LED lights) may be provided to set forth information such as battery status. Status indicator(s) 262 may, for example, indicate when battery 260 is low (RED), when the device is charging battery 260 (BLUE), and when charging of battery 260 is complete (GREEN). An on/off or power switch 280 may, for example, be provided on housing 202. A sample port or tube 290 may pass through housing 202 and has an outlet in the vicinity of chemiresistor 10a or FET 10 of sensor assembly 210.

Methodologies/circuits for sensing changes in (chemiresistor) sensor resistance in device or system 200 are shown in FITS. 9D through 9F. FIG. 9D illustrates a simple voltage divider configuration of resistors, where a change in resistance is converted to a change in voltage. In the voltage divider network, one resistor (R) is a fixed value, and the other resistance (RCNT) is variable, wherein RCNT represents the sensor resistance. In a number of embodiments, an analog-to-digital converter (ADC) is the input port of a digitizing device, such as a microcontroller/microprocessor. Sensing changes in RCNT is easier when the resistance change results in a larger voltage change. The point where the largest voltage change occurs will be when R equals the nominal sensor resistance before a measurement is taken (for example, R=RCNT).

In a number of embodiments, the resistor network in device 200 is a Wheatstone bridge as illustrated in FIG. 9E, which uses the same principle of voltage division described above but increases the resistance-sensing accuracy with a more complex resistor configuration as illustrated in FIG. 9D. Three of the resistor values are known. The fourth resistor value can be calculated from a measurement of the differential voltage between the centers of each “leg” of the bridge, labeled in FIG. 9E as Vwhtstn. Sensor 10, 10a forms the bottom half of one leg of the bridge. FIG. 9F illustrates an embodiment of electronic circuitry for system 200.

In summary, nanosensor-based (for example, sc-SWCNT-based) FET biosensor functionalized with an antibody fragment such as the reduced antibody fragment (for example, with norfentanyl reduced antibody) were developed for the sensitive detection of an antigen such as fentanyl, norfentanyl (the primary inactive metabolite of fentanyl), and other antigens. Sc-SWCNTs provide a versatile platform for chemical functionalization. Representative FET sensors adopting both the direct coupling approach and AuNP approach for the attachment of norfentanyl reduced antibody demonstrate outstanding sensing capabilities for the detection of norfentanyl, reaching limit of detection at the fg/mL level. In representative embodiments hereof, reducing the norfentanyl antibody and, for example, utilizing the free thiol groups on the half antibody fragment for the oriented attachment on AuNP-SWCNTs, further optimizes the sensing matrix. Without limitation to any mechanism, by reducing the distance between the binding sites and the sensor surface (through use of antibody fragments rather than the full antibody), the calibration sensitivity of the biosensor was enhanced by 69.6% in the case of representative reduced antibodies. As clear to those skilled in the art, the use of reduced antibodies and/or other antibody fragments in nanostructure-based sensor media may be used in the detection of antigens other than fentanyl or norfentanyl via immobilization of the appropriate/corresponding antibody fragment using various immobilization techniques/reactions.

The studies hereof further demonstrated successful application of sensor fabrication with a flexible FET electrode with a portable sensing setup, showing great potential for developing a portable device for on-site detection of fentanyl exposure with improved sensitivity. Overall, the development of effective biosensors for the detection of opioids and their metabolites is crucial for the monitoring of opioid abuse and for the management of opioid-related health issues. The norfentanyl reduced antibody-functionalized sc-SWCNT-based FET biosensors hereof further provide a platform technique for multiplexed sensing for other opioid byproducts to help fight in the opioid crisis.

### EXPERIMENTAL EXAMPLES

Device fabrication. Interdigitated gold electrodes (IDEs) were patterned on a Si/SiO₂ substrate using photolithography, forming 10 μm channels. Semiconducting single-walled carbon nanotubes (IsoSol-S100, Raymor Industries Inc.) were prepared at 0.02 mg/mL in toluene and deposited between gold electrodes via dielectrophoresis (DEP) with an ac frequency of 100 kHz, applied bias voltage of 10 V, and bias duration of 120 s. The devices were annealed at 200° C. for 1 hour before use.

For FET devices fabricated via the direct coupling approach, the sc-SWCNTs were first incubated in a 50 mM/50 mM 1-ethyl-3-(3-dimethylaminopropyl)carbodiimide (EDC)/N-hydroxysulfosuccinimide (sulfo-NHS) solution for 30 min to activate the carboxylic acid groups. Norfentanyl antibody (10 μL, 117 μg/mL in PBS buffer) was then introduced on the sc-SWCNTs surface directly after activation and incubated overnight at 4° C.

For FET devices fabricated via gold nanoparticle (AuNP) approach, gold nanoparticles were deposited on sc-SWCNTs via bulk electrolysis using a CH Instruments electrochemical analyzer in a three-electrode setup (1 M Ag/AgCl reference electrode, Pt counter electrode, and IDEs as working electrodes) from a HAuCl₄ solution (1 mM in 0.1 M HCl). The deposition voltage was set at −0.2 V and applied for 30 s. Norfentanyl antibody solution was then immobilized on the device surface by an incubating overnight at 4° C.

After the attachment of norfentanyl antibody, a blocking buffer (0.1% Tween 20 and 4% polyethylene glycol in PBS) was applied to the device surface to block unreacted surfaces.

Scanning electron microscopy (SEM). Scanning electron microscopy was performed on a Si/SiO₂ chip using a ZEISS Sigma 500 VP instrument.

Atomic force microscopy (AFM). AFM data was collected using Bruker multimode 8 AFM system with a Veeco Nanoscope IIIa controller in tapping mode. AFM image and height profiles were processed and obtained in Gywddion.

X-ray photoelectron spectroscopy (XPS). X-ray photoelectron spectroscopy data was generated on a Thenno ESCALAB 250 Xi XPS using monochromated A1 Kα X-rays as the source. A 650-micrometer spot size was used, and the samples were charge compensated using an electron flood gun.

Raman spectroscopy. Raman characterization of the devices was performed using a XplorA Raman-AFM/TERS system. Radial breathing mode (RBM) region was recorded using 785 nm (100 mW) excitation laser operating at 1% power. D and G peak region was recorded using 638 nm (24 mW) excitation laser operating at 1% power.

Cyclic voltammetry (CV). Norfentanyl sensing using sc-SWCNT-based FET biosensors via cyclic voltammetry was conducted using a CH Instruments electrochemical analyzer. A three-electrode configuration was used, where sc-SWCNT acted as the working electrode, Ag/AgCl electrode was used as the reference electrode, and Pt wire was used as the auxiliary electrode. The CV experiments were performed by sweeping the voltage from −0.6 V to +0.6 V for 10 cycles with a scan rate of 20 mV/s.

FET measurements. A Metrohm DropSens μStat-i 400 potentiostat was used for all FET measurements. FET transfer characteristics were measured employing a liquid-gated FET device configuration. A 1 M Ag/AgCl reference electrode was used as the gate electrode. Source-drain current (I_{d}) was collected by sweeping the gate voltage (V_{g}) from +0.6 V to −0.6 V while keeping the source-drain voltage at 50 mV. The gating media was 0.001×PBS.

A series of norfentanyl solutions were prepared from 1.0 ag/mL to 1.0 μg/mL. For calibration samples, the solutions were prepared in 1× phosphate buffered saline (PBS). For synthetic urine samples, 1× synthetic urine was first prepared according to Table S1. The norfentanyl solutions were prepared in 1× synthetic urine, and followed by 1:10, 1:100 and 1:1000 dilution with 1×PBS. All norfentanyl samples were tested from the lowest to the highest concentrations.

For each device, FET transfer characteristics were first collected in a blank sample, which will be used as a baseline. Next, 10 μL of each norfentanyl sample was added to the surface of the device and incubated for 10 min. After the incubation, the device was washed/rinsed with nanopure water to remove the unbound sample, and FET transfer characteristics were collected in the gating media in order to keep the same ionic strength for all FET measurements. See U.S. Patent Publication No. US2022/0365078, the disclosure of which is incorporated herein by reference.

As described above, the methods hereof may include exposing the sensor to the fluid sample for a period of time, and subsequent to exposing the sensor to the fluid sample, washing the sensor one or more times with a liquid of known ionic strength. After washing the sensor, an output of the sensor is measured with the liquid of known ionic strength over or covering the sensor medium/sensor. The liquid having a known ionic strength may, for example, be chosen to have an ionic strength that is less than the fluid sample to increase sensitivity compared to output measured in the presence of the fluid sample. In a number of embodiments, the sensor is washed a plurality of times. As described above, such washing may, for example, remove unbound species. In a number of embodiments, the liquid is a purified water. In a number of embodiments, the liquid (for example, purified water) has resistivity greater than or equal to 18.2 MΩ·cm.

The relative response (R) of each FET device was calculated as R=ΔI/I₀ at V_{g}=−0.5 V, where ΔI=I_{d}−I₀, and I₀ is the drain current in blank sample (baseline) before analyte exposure at applied gate voltage of −0.5 V. The calibration curve was plotted by reporting the averaged relative conductance of all devices tested with standard error as error bars at each concentration. The number of devices (n) tested for each experiment was specified in the figure. Calibration sensitivity was defined as the slope of the linear region on the calibration curve. The linear region was located by fitting the calibration curve using a Logistic model.

Reduction of norfentanyl antibody. The Reaction Buffer was prepared by adding 10 mM ethylenediaminetetraacetic acid (EDTA) to 1×PBS. Six milligrams of 2-mercaptoethylamine·HCl (2-MEA) was dissolved in 100 μL Reaction Buffer, then 5 μL of this 2-MEA solution was immediately added to 50 μL of norfentanyl antibody solution (1.03 mg/mL) in PBS. The reaction mixture was kept in an incubator at 37° C. for 90 min. After the reaction, a buffer exchange was performed using a desalting column to remove the 2-MEA from the reduced antibody. The final solution with the reduced norfentanyl antibody was aliquoted and frozen for further use.

Reduction of fentanyl antibody. Fentanyl antibody was purchased from commercial sources. The reduced fentanyl antibody was prepared using the same method described for reduction of norfentanyl antibody.

Reduced fentanyl antibody device fabrication. To fabricate the reduced fentanyl antibody (r-fentanyl-ab)-functionalized Au-SWCNT FET sensor, the r-fentanyl-ab was prepared at around 100 μg/mL in 1× phosphate buffered saline (PBS) and was added to the sensor chip to allow adsorption of r-fentanyl-ab on AuNPs. The Au-SWCNT FET devices were incubated with the r-fentanyl-ab solution for at least 12 hours at 4° C. to ensure maximum immobilization.

Reduced fentanyl antibody sensing studies. Sensing experiments were performed using the same method described above for norfentanyl sensing. The fentanyl sensing results of the r-fentanyl-ab Au-SWCNT FET sensor are shown in the FIG. 8.

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

What is claimed is:

1. A sensor device for detecting an antigen in a fluid sample, comprising:
a sensor comprising a substrate and a sensor medium on the substrate, the sensor medium including a plurality of single-walled carbon nanotubes having an enriched semiconducting content and one or more antibody fragments immobilized on the plurality of single-walled carbon nanotubes, each of the one or more antibody fragments comprising an active binding site for the antigen, and electronic circuitry including at least one measurement system in operative connection with the sensor to measure a variable providing a measure of change in at least one property of the sensor medium which is dependent upon the presence of the antigen.

2. The sensor device of claim 1 wherein the one or more antibody fragments are covalently attached to at least a portion of the plurality of single-walled carbon nanotubes or are attached to at least a portion of a plurality of gold nanoparticles immobilized upon the plurality of single-walled carbon nanotubes.

3. The sensor device of claim 1 wherein the plurality of single-walled carbon nanotubes has an enriched semiconducting content of at least 90%.

4. The sensor device of claim 2 wherein the one or more antibody fragments are attached to at least a portion of the plurality of gold nanoparticles immobilized upon the plurality of single-walled carbon nanotubes.

5. The sensor device of claim 4 wherein the plurality of single-walled carbon nanotubes has an enriched semiconducting content of at least 99%.

6. The sensor device of claim 1 wherein the variable measured is an electrical property change.

7. The sensor device of claim 1 wherein the antigen is a drug or a metabolite of a drug.

8. The sensor device of claim 7 wherein the drug is fentanyl, cocaine, morphine, hydrocodone, codeine, and tetrahydrocannabinol (THC).

9. The sensor device of claim 7 wherein the metabolite of the drug is norfentanyl and 6-Acetylmorphine.

10. The sensor device of claim 1 wherein the sensor is incorporated within a field effect transistor circuit of the electronic circuitry and a liquid of known and low ionic strength is used as a liquid gate.

11. The sensor device of claim 1 wherein the sensor is incorporated within a field effect transistor circuit of the electronic circuitry, the field effect transistor circuitry comprises a gold gate, and the one or more antibody fragments are also immobilized on the gold gate.

12. The sensor device of claim 1 wherein the sensor medium is maintained in a liquid phase.

13. The sensor device of claim 1 wherein the one or more antibody fragments comprise reduced antibody fragments.

14. A sensor, comprising: a substrate and a sensor medium on the substrate, the sensor medium comprising a plurality of single-walled carbon nanotubes having an enriched semiconducting content and one or more antibody fragments immobilized on the plurality of single-walled carbon nanotubes, each of the one or more antibody fragments comprising an active binding site for an antigen to be detected, wherein at least one property of the sensor medium is dependent upon the presence of the antigen.

15. The sensor of claim 14 wherein the one or more antibody fragments are covalently attached to at least a portion of the plurality of single-walled carbon nanotubes or are attached to at least a portion of a plurality of gold nanoparticles immobilized upon the plurality of single-walled carbon nanotubes.

16. The sensor of claim 14 wherein the plurality of single-walled carbon nanotubes has an enriched semiconducting content of at least 90%.

17. The sensor of claim 15 wherein the one or more antibody fragments are attached to at least a portion of the plurality of gold nanoparticles immobilized upon the plurality of single-walled carbon nanotubes.

18. The sensor of claim 14 wherein the antigen is a drug or a metabolite of a drug.

19. The sensor of claim 18 wherein the drug is fentanyl, cocaine, morphine, hydrocodone, codeine, and tetrahydrocannabinol (THC).

20. The sensor of claim 18 wherein the metabolite of the drug is norfentanyl and 6-Acetylmorphine.

21. A method of detecting an antigen in a fluid sample, comprising:
providing a sensor device including a sensor including a substrate and a sensor medium on the substrate, the sensor medium including a plurality of single-walled carbon nanotubes having an enriched semiconducting content and one or more antibody fragments, each of the one or more antibody fragments comprising an active binding site for the antigen, and electronic circuitry including at least one measurement system in operative connection with the sensor to measure a variable providing a measure of change in at least one property of the sensor medium which is dependent upon the presence of the antigen;
exposing the sensor to the fluid sample for a period of time; and
measuring an output of the sensor with a liquid of known ionic strength over the sensor medium.
